(19) United States
(12) Patent Application Publication
(10) Pub. No.: US 2014/0147516 A1
(43) Pub. Date: May 29, 2014

(54) POLYMORPHISMS PREDICTIVE OF PLATINUM-COORDINATING COMPOUND-INDUCED OTOTOXICITY

(71) Applicant: The University of British Columbia, Vancouver (CA)

(72) Inventors: Michael R. Hayden, Vancouver (CA); Bruce Carleton, West Vancouver (CA); Colin Ross, Burnaby (CA)

(73) Assignee: The University of British Columbia, Vancouver (CA)

(21) Appl. No.: 14/095,735

(22) Filed: Dec. 3, 2013

Related U.S. Application Data
(63) Continuation of application No. 12/937,343, filed on Dec. 30, 2010.
(60) Provisional application No. 61/136,567, filed on Sep. 15, 2008.
(60) Provisional application No. 61/202,754, filed on Apr. 1, 2009.

(51) Int. Cl. C12Q 1/68

(52) U.S. Cl. CPC C12Q 1/6883
USPC 424/649; 514/262.1; 514/99; 514/492

(57) ABSTRACT

Methods of determining a subject's ototoxicity risk from administration of a pharmacotherapeutic compound having an ototoxicity risk, methods of administering a pharmacotherapeutic compound having an ototoxicity risk and oligonucleotides, peptide nucleic acids, arrays, and addressable collections for performing embodiments of the methods are provided herein.

### CROSS-REFERENCE TO RELATED APPLICATIONS

This application is a continuation of U.S. patent application Ser. No. 12/937,343, filed on Oct. 11, 2010, which is a 371 filing of International Application No. PCT/CA2009/000479, filed on Apr. 14, 2009, which claims priority to and the benefit of U.S. Provisional Patent Application No. 61/071,058, filed on Apr. 10, 2008, U.S. Provisional Patent Application No. 61/136,567, filed on Sep. 15, 2008, and U.S. Provisional Patent Application No. 61/202,754, filed on Apr. 1, 2009, the entire contents of all of which are hereby incorporated by reference.

### INCORPORATION BY REFERENCE

The material in the text file entitled “12937343SEQLISTING”, created on Dec. 30, 2010 and being 12,000 bytes in size, is herein incorporated by reference in its entirety.

### FIELD OF THE INVENTION

This invention relates to the field of genetic markers for adverse drug reactions. More specifically, methods and compositions useful for identifying individuals that may be at risk for an adverse drug reaction.

### BACKGROUND

Adverse drug reactions (ADRs) are a significant cause of illness, hospitalization and death for both children and adults in the Western world (Lazarou et al. 1998. JAMA 279:1200-05; Pirmohamed et al. 2004. BMJ 329:15-19). Estimates suggest that 15% of hospitalized children experience an ADR. Those that do survive the ADR may be left disabled (Mitchell et al., 1988. Pediatrics 82:24-9; Martinez-Mir et al., 1999. Br J Clin Pharmacol 47:681-88).

Many approved drugs used in children are untested in pediatric populations. While it is known that children metabolize drugs differently than adults, in many cases pediatric dosage forms are not available. This is of particular concern with pharmacotherapy drugs which may frequently be supplied as a single-dose package, and in combination with other agents, excipients and the like. Pediatric populations also represent a more varied population, and this increased variability may be due to developmental differences in the normal expression of drug metabolism genes.

Genetic factors are involved in variability in drug response—ranging from 20-95% in some studies. Age, sex, body weight, health, medical history and the like may be accounted for, but patient genotype is largely an unknown factor (Evans et al., 2003. NEJM 348:538-49; Weinshilboum, 2003. NEJM 348:529-537).

Two major classes of drugs currently in clinical use can cause permanent hearing loss. Aminoglycoside antibiotics have a major role in the treatment of life-threatening infections, and platinum-based pharmacotherapeutic compounds are highly effective in the treatment of malignant disease. Both are reported to damage the hair cells of the inner ear, resulting in functional deficits.

Aminoglycoside antibiotics were developed in 1944 to treat gram-negative bacteria that were not responsive to conventional antibiotics, such as penicillin. These compounds can be characterized by amino sugars that have glycosidic linkages. Subsequently, a number of similar compounds have been developed and are still commonly used. However, their clinical use is limited by toxic side effects that include cochlear toxicity, vestibular toxicity and nephrotoxicity. The aminoglycoside antibiotics include, for example, streptomycin, kanamycin, tobramycin, neomycin, gentamicin, amikacin and netilmicin. All display ototoxicity but vary in their preferential damage to the cochlea or vestibule.

Platinum-coordinating compounds are used as cytotoxic agents in pharmacotherapeutic protocols for a variety of neoplasms in both children and adults. For example, cisplatin may be used in the treatment of solid tumors including those of the lung, testicular, ovarian, breast, bladder and head-and-neck. In children, cisplatin is used in the treatment of some cancers, including CNS tumors, hepatoblastoma, neuroblastoma and osteosarcoma. Other platinum-coordinating compounds include carboplatin, oxaliplatin, tetraplatin, ormiplatin, iproplatin, the orally available satraplatin (Kelland, 2000. Expert Opin Investig Drugs 9: 1373-82), nedaplatin, eptaplatin, lobaplatin, picoplatin, miboplatin, and aroplatin. The pharmacotherapeutic effect of platinum-coordinating compounds may result from DNA binding and crosslinking in rapidly dividing cells.

Cisplatin normally binds thiol-containing compounds and purines, especially guanine, and exerts its cytotoxic effect by forming intra-strand and inter-strand DNA cross-links, causing cell death in rapidly dividing cells. TPMT can methylate and inactivate exogenous thiopurine compounds, such as the metabolites of azathioprine (Weinshilboum et al., 2006. Cell Mol Neurobiol 26: 539-61; Weinshilboum et al., 1980. Am J Hum Genet. 32: 651-62). It is possible that a loss of TPMT enzyme activity could also reduce the inactivation of cisplatin-purine compounds, thereby increasing the efficiency of cisplatin cross-linking, and increasing cisplatin toxicity.

S-adenosyl methionine (SAM) substantially increases cisplatin-induced toxicity in cisplatin-treated mice (Ochoa et al., 2009. Arch Med Res. 40: 54-85). Administration of SAM alone is not toxic, and administration of cisplatin alone exhibits moderate toxicity, while administration of SAM and cisplatin dramatically increase cisplatin toxicity, as monitored by renal dysfunction (creatinine and BUN). These results suggest that cisplatin-induced ototoxicity could be related to increased levels of SAM. TPMT and COMT are methyltransferases dependent on SAM methyl donor substrate in the methionine pathway (Weinshilboum et al., 2006. Cell Mol Neurobiol 26: 539-61; Weinshilboum et al., 1980. Am J Hum Genet. 32: 651-62). COMT-like enzyme activity is involved in auditory function in mice and humans (Ahmed et al., 2008. Nat Genet. 40: 1335-40; Du et al., 2008. Proc Natl Acad Sci USA. 105: 14609-14).

One strategy to protect the inner ear from ototoxicity is the administration of antioxidant drugs to provide upstream protection and block the activation of cell-death sequences. Downstream prevention involves the interruption of the cell-death cascade that has already been activated, to prevent apoptosis. Challenges and opportunities exist for appropriate drug delivery to the inner ear and for avoiding interference with the therapeutic efficacy of both categories of ototoxic drugs.

Ototoxicity is a serious problem in patient populations receiving platinum-coordinating compounds, particularly pediatric patients (Kushner et al., 2006. Cancer 107:417-22). Platinum-coordinating compound-induced ototoxicity may range from tinnitus to irreversible hearing impairment. Increased and cumulative dose, nature of the particular coordination complex, administration route, age and prior radiation treatment are known to affect onset and severity of ototoxicity, but the incidence may be inconsistent (Stohr et al., 2005 and references therein). Oxidative stress has been implicated as a possible cause of cisplatin-induced ototoxicity (Peters et al., 2000. Anticancer Drugs. 11:639-43).

Cisplatin has been described as one of the most ototoxic drugs in clinical use, causing severe, permanent, bilateral hearing loss in 10-25% of adult patients, 50% of patients receiving high doses (>400 mg/m²), and 41-61% of children (Li et al., 2004. Eur. J. Cancer 40: 2445-51; Coradini et al., 2007. J Pediatr Hematol Oncol 29: 355-60; Knight et al., 2005. J Clin Oncol 23: 8588-96; Kushner et al., 2006. Cancer 107: 417-22; Blakley et al., 1994. Arch Otolaryngol Head Neck Surg 120: 541-46). In children, hearing loss is perhaps most profound because even mild losses of hearing considerably increase a child's risk of learning difficulties and social-emotional problems (Knight et al., 2005. J Clin Oncol 23: 8588-96; Bess et al., 1998. Ear Hear 19: 339-54). Hearing tests are routinely administered before, during and after treatment with cisplatin. Despite significant inter-individual variation in ototoxicity in patients receiving similar doses of cisplatin, cisplatin-ototoxicity frequently leads to dose reduction and premature termination of cisplatin treatment.

Genotype has been shown to alter response to therapeutic interventions. Genentech's HERCEPTIN® was not effective in its overall Phase III trial but was shown to be effective in a genetic subset of subjects with human epidermal growth factor receptor 2 (HER2)-positive metastatic breast cancer. Similarly, Novartis' GLEEVEC® is only indicated for the subset of chronic myeloid leukemia subjects who carry a reciprocal translocation between chromosomes 9 and 22.

Due to inter-individual variation in ototoxicity in patients receiving similar doses of cisplatin has led to speculation that some patients may have polymorphisms associated with cisplatin-ototoxicity susceptibility (Ekborn at al., 2000. Hear Res 140: 38-44). Huang and colleagues identified a variety of genetic variants that were associated with cisplatin-induced cytotoxicity in defined patient populations (Huang et al. 2007. Am. J. Hum. Genetics 81:427-437). Hayden and colleagues identified genetic variants that were associated with the likelihood of developing ototoxicity in response to therapeutic intervention (Hayden et al., 2008. WO08/058,395 A1). Riedemann and colleagues identify polymorphisms in the megalin gene that were associated with cisplatin-induced ototoxicity (Riedemann et al. 2008. The Pharmacogenomics Journal. 8:23-28). Polymorphisms in genes that encode glutathione-S-transferase enzymes have also been linked to cisplatin-induced hearing impairment (Oldenburg et al. 2007. J. Clin Oncol. 25:708-14).

### SUMMARY

This invention is based in part on the identification of the particular nucleotides (alleles) or genotypes at the site of a given single nucleotide polymorphism (SNP) which are associated with a increased likelihood of ototoxicity (‘risk genotype’) or a decreased likelihood of ototoxicity (‘decreased risk genotype’).

This invention is also based in part on the surprising discovery that rs1994798; rs2410556; rs4242626; rs7867504; rs11140511; rs4877831; rs7853758; rs740150; rs6464431; rs12201199; rs1142345 (formerly rs16880254); rs1800460; rs9332377; rs207425; rs3768293; rs3101826; and rs1472408 SNPs alone or in combination are useful in predicting a subject's risk of ototoxicity following administration of a pharmacotherapeutic having an ototoxicity risk, whereby the subjects having a decreased risk genotype are less likely to experience ototoxicity and subjects having a risk genotype are more likely to experience ototoxicity from the same treatment. Furthermore, this invention is also based on the surprising result that any one or more of the following SNPs: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; and SNPs in linkage disequilibrium (LD) thereto, may be taken in combination with rs4646316 to increase the predictive values.

In accordance with one embodiment, there is provided a method of determining a subject's ototoxicity risk from administration of a pharmacotherapeutic compound having an ototoxicity risk, the method including (a) determining the identity of one or more of the following polymorphic sites in the subject: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211; and (b) assessing the subject's ototoxicity risk based on the identity of the one or more polymorphic sites.

In accordance with another embodiment, there is provided a method of method of selecting a therapeutic regimen for a subject, the therapeutic regimen comprising one or more pharmacotherapeutic compounds having an ototoxicity risk, the method including: (a) determining the identity of one or more of the following polymorphic sites in the subject: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211; and (b) assessing the subject's ototoxicity risk based on the identity of the one or more polymorphic sites.

The method may further include subsequently selecting from one or more of the following treatment alternatives: (i) administering the pharmacotherapeutic compound having an ototoxicity risk; (ii) not administering the pharmacotherapeutic compound; (iii) administering an alternative therapeutic not having ototoxicity risk or a reduced risk; (iv) administering an adjunct therapy to reduce risk of ototoxicity; and (v) monitoring of the subject for signs of ototoxicity.

In accordance with another embodiment, there is provided a method of treating a subject with a pharmacotherapeutic compound having an ototoxicity risk, the method including:

(a) determining the identity of one or more of the following polymorphic sites in the subject: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211; and
(b) selecting from one or more of the treatment alternatives based on the identity at the one or more polymorphic sites:
(i) administering the pharmacotherapeutic compound having an ototoxicity risk;
(ii) administering an alternative therapeutic not having an ototoxicity risk or having a reduced ototoxicity risk;
(iii) administering an adjunct therapy to reduce risk of ototoxicity; and
(iv) monitoring of the subject for signs of ototoxicity.

In accordance with another embodiment, there is provided a use of a pharmacotherapeutic compound having an ototoxicity risk in the manufacture of a medicament for the treatment of a subject having an approved indication of the pharmacotherapeutic compound having an ototoxicity risk, wherein the subject treated has a reduced ototoxicity risk genotype at one or more of the following polymorphic sites: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211.

In accordance with another embodiment, there is provided a use of a pharmacotherapeutic compound having an ototoxicity risk for the treatment of a subject having an approved indication for the pharmacotherapeutic compound having an ototoxicity risk, wherein the subject treated has a reduced ototoxicity risk genotype at one or more of the following polymorphic sites: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211.

In accordance with another embodiment, there is provided a method of determining risk of ototoxicity for a therapeutic regimen known or suspected of being ototoxic, the method comprising: (a) determining the identity of a single nucleotide polymorphism (SNP) at one or more of the following polymorphic sites: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211, where the test subject is a candidate for administration of a pharmacotherapeutic compound having an ototoxicity risk; and (b) separating test subjects based on their risk of ototoxicity prior to administration of the pharmacotherapeutic compound.

In accordance with another embodiment, there is provided a method for selecting a group of subjects for determining the side effects of a candidate pharmacotherapeutic compound known or suspected of being ototoxic, the method comprising: (a) determining a subject's genotype for a single nucleotide polymorphism (SNP) at one or more of the following polymorphic sites: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211, for each subject, wherein a subject's genotype is indicative of the subject's risk of ototoxicity following therapeutic regimen administration; and (b) sorting subjects based on genotype or ototoxicity risk.

The approved indication may be a neoplastic disease. The approved indication may be an infection. The approved indication may be a gram negative infection.

The identity associated with ototoxicity risk or associated with decreased ototoxicity risk may be selected from one or more of: rs1994798gg; rs2410556 cc; rs4242626gg; rs7867504gg; rs11140511aa or rs11140511ac or rs11140511 cc; rs4877831gg or rs4877831gc; rs7853758gg or rs7853758ga or rs7853758aa; rs740150gg or rs740150ga; rs6464431aa or rs6464431 at; rs12201199aa or rs12201199 at; rs1142345gg or rs1142345ga rs1800460aa or rs1800460ag; rs3101826aa or rs3101826ag or rs3101826gg; rs9332377aa or rs9332377ag; rs207425aa; rs3768293 cc; and rs1472408gg.

The determining the identity of the one or more of the polymorphic sites may be by one or more of the following techniques: (a) restriction fragment length analysis; (b) sequencing; (c) micro-sequencing assay; (d) hybridization; (e) invader assay; (f) gene chip hybridization assays; (g) oligonucleotide ligation assay; (h) ligation rolling circle amplification; (i) 5′ nuclease assay; (j) polymerase proofreading methods; (k) allele specific PCR; (l) matrix assisted laser desorption ionization time of flight (MALDI-TOF) mass spectroscopy; (m) ligase chain reaction assay; (n) enzyme-amplified electronic transduction; (o) single base pair extension assay; and (p) reading sequence data.

The pharmacotherapeutic compound having an ototoxicity risk may be a platinum-coordinating compound. Alternatively, the pharmacotherapeutic compound having an ototoxicity risk may be an aminoglycoside. Alternative pharmacotherapeutic compounds having an ototoxicity risk may be selected from furosemide and vincristine.

The platinum-coordinating compound may be selected from one or more of the following: cisplatin; carboplatin; oxaliplatin; tetraplatin; ormiplatin; iproplatin; satraplatin; nedaplatin; picoplatin; eptaplatin; miboplatin; sebriplatin; lobaplatin; and aroplatin. The platinum-coordinating compound may be cisplatin.

The aminoglycoside may be selected from streptomycin, kanamycin, tobramycin, neomycin, gentamicin, amikacin and netilmicin.

The method may further include obtaining a sample from the subject prior to determining the identity of the one or more polymorphic sites in the subject. The method may further include administering the candidate pharmacotherapeutic compound to the subjects or a subset of subjects and assessing the degree of hearing loss in each subject. The method may further include comparing the degree of hearing loss in response to the candidate drug based on genotype of the subject.

The alternative therapeutic not having ototoxicity risk or a reduced risk may be selected from any one or more of the following: oxaliplatin, carboplatin, and a liposomal formulation of the platinum-coordinating compound having an ototoxocity risk. The adjunct therapy to reduce risk of ototoxicity may include the administration of an otoprotectant. The otoprotectant may be selected from any one or more of the following compounds: sodium thiosulfate; ebselen; d-methionine; glutathione ester; diethyldithiocarbamate; amifostine; tiopronin; α-tocopherol; salacylate; aminoguanidine; trolox; Z-DEVD-fluoromethyl ketone; ZLEKD-fluoromethyl ketone; 2-chloro-N-cyclopentyladenosine; pifithrin; α-lipoic acid; deferoxamine; 2,2′-dipyridyl; salicylate; 2,3-dihydroxybenzoate; dexamethasone; TRANSFORMING GROWTH FACTOR-β1; GLIAL-CELL-DERIVED NEUROTROPHIC FACTOR; ethacrynic acid; CEP1347; and minocycline.

Alternatively, the methods described herein may further include determining the identity of rs4646316 in combination with any one or more of the polymorphisms set out above. Furthermore, the overall ability to correctly identify ototoxicity risk based on genotype may be improved by combining rs12201199 and rs9332377, or to combine rs12201199 and rs4646316, or to combine rs12201199 and rs207425, or to combine rs4646316 and rs9332377, or to combine rs4646316 and rs207425, or to combine rs9332377 and rs207425, or to combine rs12201199, rs4646316, and rs9332377, or to combine rs12201199, rs4646316, and rs207425, or to combine rs4646316, rs9332377, and rs207425, or to combine rs12201199, rs4646316, rs9332377, and rs207425.

In accordance with another embodiment, there are provided two or more oligonucleotides or peptide nucleic acids of about 10 to about 400 nucleotides that hybridize specifically to a sequence contained in a human target sequence consisting of a subject's ototoxicity associated gene sequence, a complementary sequence of the target sequence or RNA equivalent of the target sequence and wherein the oligonucleotides or peptide nucleic acids are operable in determining the presence or absence of two or more polymorphism(s) in the ototoxicity associated gene sequence selected from one or more of the following polymorphic sites: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto selected from one or more of the following: rs12485043, rs9617857, rs9618725, rs6756897, rs11260822, rs12401559, rs12405694, rs12408442, rs12408813, rs1566145, rs2230597, rs2863841, rs3820609, rs6603867, rs6603883, rs6678616, rs4646312, rs740601, rs2239393, rs4680, rs476235, rs12199060, rs10949481, rs6908777, rs11964408, rs11121828, rs12404124, rs198391, rs198393, rs198399, rs198401, rs198406, rs198408, rs4845882, rs4846049, rs4846052, rs4846054, rs503040, rs535107, rs6541003, rs6697244, rs7538516, rs7036569, rs17426961, rs4585823, rs17427184, rs7861242, rs4877837, rs10868141, rs10868142, rs10123041, rs9792674, rs4877838, rs10746739, rs12005041, rs7863627, rs4877839, rs4877841, rs4877842, rs10780663, rs7029691, rs4877844, rs17336552, rs10122651, rs4877829, rs4877832, rs7849745, rs11140481, rs7857113, rs7857379, rs7873208, rs2184747, rs7853066, rs7047315, rs10868137, rs885004, rs4877836, rs11973494, rs6977672, rs41715, and rs2284211.

In accordance with another embodiment, there are provided two or more oligonucleotides or peptide nucleic acids selected from the group:

(a) an oligonucleotide or peptide nucleic acid that hybridizes under high stringency conditions to a nucleic acid molecule comprising SEQ ID NO:1 having a C at position 201 but not to a nucleic acid molecule comprising SEQ ID NO:1 having a T at position 201;
(b) an oligonucleotide or peptide nucleic acid that hybridizes under high stringency conditions to a nucleic acid molecule comprising SEQ ID NO:1 having a T at position 201 but not to a nucleic acid molecule comprising SEQ ID NO:1 having a C at position 201;
(c) an oligonucleotide or peptide nucleic acid that hybridizes under high stringency conditions to a nucleic acid molecule comprising SEQ ID NO:2 having a C at position 201 but not to a nucleic acid molecule comprising SEQ ID NO:2 having a T at position 201;
(d) an oligonucleotide or peptide nucleic acid that hybridizes under high stringency conditions to a nucleic acid molecule comprising SEQ ID NO:2 having a T at position 201 but not to a nucleic acid molecule comprising SEQ ID NO:2 having a C at position 201;
(e) an oligonucleotide or peptide nucleic acid that hybridizes under high stringency conditions to a nucleic acid molecule comprising SEQ ID NO:3 having a C at position 201 but not to a nucleic acid molecule comprising SEQ ID NO:3 having a T at position 201;
(f) an oligonucleotide or peptide nucleic acid that hybridizes under high stringency conditions to a nucleic acid molecule comprising SEQ ID NO:3 having a T at position 201 but not to a nucleic acid molecule comprising SEQ ID NO:3 having a C at position 201;
(g) an oligonucleotide or peptide nucleic acid that hybridizes under high stringency conditions to a nucleic acid molecule comprising SEQ ID NO:4 having a C at position 201 but not to a nucleic acid molecule comprising SEQ ID NO:4 having a T at position 201;
(h) an oligonucleotide or peptide nucleic acid that hybridizes under high stringency conditions to a nucleic acid molecule comprising SEQ ID NO:4 having a T at position 201 but not to a nucleic acid molecule comprising SEQ ID NO:4 having a C at position 201;
(i) an oligonucleotide or peptide nucleic acid that hybridizes under high stringency conditions to a nucleic acid molecule comprising SEQ ID NO:5 having an A at position 201 but not to a nucleic acid molecule comprising SEQ ID NO:5 having a C at position 201;
(j) an oligonucleotide or peptide nucleic acid that hybridizes under high stringency conditions to a nucleic acid molecule comprising SEQ ID NO:5 having a C at position 201 but not to a nucleic acid molecule comprising SEQ ID NO:5 having an A at position 201;
(k) an oligonucleotide or peptide nucleic acid that hybridizes under high stringency conditions to a nucleic acid molecule comprising SEQ ID NO:6 having a C at position 201 but not to a nucleic acid molecule comprising SEQ ID NO:6 having a G at position 201;
(l) an oligonucleotide or peptide nucleic acid that hybridizes under high stringency conditions to a nucleic acid molecule comprising SEQ ID NO:6 having a G at position 201 but not to a nucleic acid molecule comprising SEQ ID NO:6 having a C at position 201;
(m) an oligonucleotide or peptide nucleic acid that hybridizes under high stringency conditions to a nucleic acid molecule comprising SEQ ID NO:7 having an A at position 201 but not to a nucleic acid molecule comprising SEQ ID NO:7 having a G at position 201;
(n) an oligonucleotide or peptide nucleic acid that hybridizes under high stringency conditions to a nucleic acid molecule comprising SEQ ID NO:7 having a G at position 201 but not to a nucleic acid molecule comprising SEQ ID NO:7 having an A at position 201;
(o) an oligonucleotide or peptide nucleic acid that hybridizes under high stringency conditions to a nucleic acid molecule comprising SEQ ID NO:8 having a T at position 201 but not to a nucleic acid molecule comprising SEQ ID NO:8 having a C at position 201;
(p) an oligonucleotide or peptide nucleic acid that hybridizes under high stringency conditions to a nucleic acid molecule comprising SEQ ID NO:8 having a C at position 201 but not to a nucleic acid molecule comprising SEQ ID NO:8 having a T at position 201;
(q) an oligonucleotide or peptide nucleic acid that hybridizes under high stringency conditions to a nucleic acid molecule comprising SEQ ID NO:9 having a T at position 201 but not to a nucleic acid molecule comprising SEQ ID NO:9 having an A at position 201;
(r) an oligonucleotide or peptide nucleic acid that hybridizes under high stringency conditions to a nucleic acid molecule comprising SEQ ID NO:9 having an A at position 201 but not to a nucleic acid molecule comprising SEQ ID NO:9 having a T at position 201;
(s) an oligonucleotide or peptide nucleic acid that hybridizes under high stringency conditions to a nucleic acid molecule comprising SEQ ID NO:10 having an A at position 201 but not to a nucleic acid molecule comprising SEQ ID NO:10 having a T at position 201;
(t) an oligonucleotide or peptide nucleic acid that hybridizes under high stringency conditions to a nucleic acid molecule comprising SEQ ID NO:10 having a T at position 201 but not to a nucleic acid molecule comprising SEQ ID NO:10 having an A at position 201;
(u) an oligonucleotide or peptide nucleic acid that hybridizes under high stringency conditions to a nucleic acid molecule comprising SEQ ID NO:11 having an A at position 201 but not to a nucleic acid molecule comprising SEQ ID NO:11 having a G at position 201;
(v) an oligonucleotide or peptide nucleic acid that hybridizes under high stringency conditions to a nucleic acid molecule comprising SEQ ID NO:11 having a G at position 201 but not to a nucleic acid molecule comprising SEQ ID NO:11 having an A at position 201;
(w) an oligonucleotide or peptide nucleic acid that hybridizes under high stringency conditions to a nucleic acid molecule comprising SEQ ID NO:12 having an A at position 201 but not to a nucleic acid molecule comprising SEQ ID NO:12 having a G at position 201;
(x) an oligonucleotide or peptide nucleic acid that hybridizes under high stringency conditions to a nucleic acid molecule comprising SEQ ID NO:12 having a G at position 201 but not to a nucleic acid molecule comprising SEQ ID NO:12 having an A at position 201;
(y) an oligonucleotide or peptide nucleic acid that hybridizes under high stringency conditions to a nucleic acid molecule comprising SEQ ID NO:13 having a C at position 201 but not to a nucleic acid molecule comprising SEQ ID NO:13 having a T at position 201;
(z) an oligonucleotide or peptide nucleic acid that hybridizes under high stringency conditions to a nucleic acid molecule comprising SEQ ID NO:13 having a T at position 201 but not to a nucleic acid molecule comprising SEQ ID NO:13 having a C at position 201;
(aa) an oligonucleotide or peptide nucleic acid that hybridizes under high stringency conditions to a nucleic acid molecule comprising SEQ ID NO:14 having a C at position 201 but not to a nucleic acid molecule comprising SEQ ID NO:14 having a T at position 201;
(bb) an oligonucleotide or peptide nucleic acid that hybridizes under high stringency conditions to a nucleic acid molecule comprising SEQ ID NO:14 having a T at position 201 but not to a nucleic acid molecule comprising SEQ ID NO:14 having a C at position 201;
(cc) an oligonucleotide or peptide nucleic acid that hybridizes under high stringency conditions to a nucleic acid molecule comprising SEQ ID NO:15 having an A at position 201 but not to a nucleic acid molecule comprising SEQ ID NO:15 having a G at position 201;
(dd) an oligonucleotide or peptide nucleic acid that hybridizes under high stringency conditions to a nucleic acid molecule comprising SEQ ID NO:15 having a G at position 201 but not to a nucleic acid molecule comprising SEQ ID NO:15 having an A at position 201;
(ee) an oligonucleotide or peptide nucleic acid that hybridizes under high stringency conditions to a nucleic acid molecule comprising SEQ ID NO:16 having an A at position 201 but not to a nucleic acid molecule comprising SEQ ID NO:16 having a C at position 201;
(ff) an oligonucleotide or peptide nucleic acid that hybridizes under high stringency conditions to a nucleic acid molecule comprising SEQ ID NO:16 having a C at position 201 but not to a nucleic acid molecule comprising SEQ ID NO:16 having an A at position 201;
(gg) an oligonucleotide or peptide nucleic acid that hybridizes under high stringency conditions to a nucleic acid molecule comprising SEQ ID NO:17 having a G at position 201 but not to a nucleic acid molecule comprising SEQ ID NO:17 having an A at position 201;
(hh) an oligonucleotide or peptide nucleic acid that hybridizes under high stringency conditions to a nucleic acid molecule comprising SEQ ID NO:17 having an A at position 201 but not to a nucleic acid molecule comprising SEQ ID NO:17 having a G at position 201;
(ii) an oligonucleotide or peptide nucleic acid that hybridizes under high stringency conditions to a nucleic acid molecule comprising SEQ ID NO:18 having a T at position 201 but not to a nucleic acid molecule comprising SEQ ID NO:18 having a C at position 201;
(jj) an oligonucleotide or peptide nucleic acid that hybridizes under high stringency conditions to a nucleic acid molecule comprising SEQ ID NO:18 having a C at position 201 but not to a nucleic acid molecule comprising SEQ ID NO:18 having a T at position 201;
(kk) an oligonucleotide or peptide nucleic acid capable of hybridizing under high stringency conditions to a nucleic acid molecule comprising a first allele for a given polymorphism selected from the polymorphisms listed in TABLE 3 but not capable of hybridizing under high stringency conditions to a nucleic acid molecule comprising a second allele for the given polymorphism selected from the polymorphisms listed in TABLE 3;
(ll) an oligonucleotide or peptide nucleic acid capable of hybridizing under high stringency conditions to a nucleic acid molecule comprising the second allele for a given polymorphism selected from the polymorphisms listed in TABLE 3 but not capable of hybridizing under high stringency conditions to a nucleic acid molecule comprising the first allele for the given polymorphism selected from the polymorphisms listed in TABLE 3.

In accordance with another embodiment, there is provided an array of oligonucleotides or peptide nucleic acids attached to a solid support, the array comprising two or more of the oligonucleotides or peptide nucleic acids set out herein.

In accordance with another embodiment, there is provided a composition comprising an addressable collection of two or more oligonucleotides or peptide nucleic acids, the two or more oligonucleotides or peptide nucleic acids consisting essentially of two or more nucleic acid molecules set out in SEQ ID NO: 1-18 or compliments, fragments, variants, or analogs thereof.

The oligonucleotides or peptide nucleic acids may further include one or more of the following: a detectable label; a quencher; a mobility modifier; a contiguous non-target sequence situated 5′ or 3′ to the target sequence or 5′ and 3′ to the target sequence.

Furthermore, the oligonucleotides or peptide nucleic acids or arrays or addressable collections described herein may be contained in a kit or a commercial package. The kit or the commercial package may further comprise instructions for use.

### BRIEF DESCRIPTION OF THE DRAWINGS

FIGS. 1a and 1b show linkage disequilibrium maps of the TPMT and COMT genomic regions.

FIG. 2 shows histograms to illustrate the genotype-driven prediction of cisplatin ototoxicity.

### DETAILED DESCRIPTION

### 1. Definitions and General Information

In the description that follows, a number of terms are used extensively, the following definitions are provided to facilitate understanding of the various embodiments of the invention.

“Genetic material” includes any nucleic acid and can be a deoxyribonucleotide or ribonucleotide polymer in either single or double-stranded form.

A nucleotide represented by the symbol M may be either an A or C, a nucleotide represented by the symbol W may be either a T/U or A, a nucleotide represented by the symbol Y may be either an C or T/U, a nucleotide represented by the symbol S may be either a G or C, while a nucleotide represented by the symbol R may be either a G or A, and a nucleotide represented by the symbol K may be either a G or T/U. Similarly, a nucleotide represented by the symbol V may be A or G or C, while a nucleotide represented by the symbol D may be A or G or T/U, while a nucleotide represented by the symbol B may be G or C or T/U, and a nucleotide represented by the symbol H may be A or C or T/U.

A “polymorphic site” or “polymorphism site” or “polymorphism” or “single nucleotide polymorphism site” (SNP site) or “single nucleotide polymorphism” (SNP) as used herein is the locus or position with in a given sequence at which divergence occurs. A “polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Polymorphic sites have at least two alleles, each occurring at a frequency of greater than 1%, and may be greater than 10% or 20% of a selected population. Polymorphic sites may be at known positions within a nucleic acid sequence or may be determined to exist. Polymorphisms may occur in both the coding regions and the noncoding regions (for example, promoters, introns or untranslated regions) of genes. Polymorphisms may occur at a single nucleotide site (SNPs) or may involve an insertion or deletion as described herein. A “risk genotype” or “ototoxicity risk genotype” as used herein refers to an allelic variant (genotype) at one or more of the following polymorphic sites: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto, for the subject as described herein, as being indicative of a increased likelihood of ototoxicity following administration of a pharmacotherapeutic compound having an ototoxicity risk (for example, a platinum-coordinating compound or an aminoglycoside compound). A risk genotype may be determined for either the haploid genotype or diploid genotype, provided that at least one copy of a risk allele is present. Risk genotype may be an indication of an increased risk of ototoxicity. Subjects having one copy (heterozygotes) or two copies (homozygotes) of the risk allele are considered to have the “risk genotype” even though the degree to which the subject's risk of ototoxicity may increase more for a subject who is a homozygote as compared to a subject who is a heterozygote. Such “risk alleles” or “risk polymorphisms” may be selected from one or more of the following: rs1994798g; rs2410556g; rs4242626g; rs7867504g; rs11140511a; rs4877831g; rs7853758g; rs740150g; rs6464431a; rs12201199a; rs1142345g; rs1800460a; rs3101826a; rs9332377g; rs207425a; rs3768293a; and rs1472408a; or a polymorphic site in linkage disequilibrium thereto (risk alleles given for the forward strand or top strand). Ototoxicity risk genotypes may be selected from one or more of the following: rs1994798gg; rs2410556 cc; rs4242626gg; rs7867504gg; rs11140511aa or rs11140511ac; rs4877831gg or rs4877831gc; rs7853758gg or rs7853758ga; rs740150gg or rs740150ga; rs6464431aa or rs6464431 at; rs12201199aa or rs12201199 at; rs1142345gg or rs1142345ga; rs1800460aa or rs1800460ag; rs3101826aa or rs3101826ag; rs9332377aa or rs9332377ag; or rs207425aa; rs3768293aa or rs3768293ac; and rs1472408aa or rs1472408ag.

A “decreased risk allele” or “decreased risk genotype” or “reduced risk genotype” or “decreased ototoxicity risk genotype” as used herein refers to an allelic variant (genotype) at one or more of the following polymorphic sites: rs1994798; rs2410556; rs4242626; rs7867504; rs11140511; rs4877831; rs7853758; rs740150; rs6464431; rs12201199; rs1142345; rs1800460; rs3101826; rs9332377; rs207425; rs3768293; and rs1472408; or a polymorphic site in linkage disequilibrium thereto, for the subject as described herein, as being indicative of a decreased likelihood of ototoxicity following administration of a platinum-coordinating compound. “Decreased risk alleles” or “reduced risk genotypes” or “reduced risk polymorphisms” may be selected from one or more of the following: rs1994798a; rs2410556a; rs4242626a; rs7867504a; rs11140511c; rs4877831c; rs7853758a; rs740150a; rs6464431t; rs12201199t; rs1142345a; rs1800460g; rs3101826g; rs9332377a; rs207425g; rs3768293c; and rs1472408g; or a polymorphic site in linkage disequilibrium thereto. “Decreased ototoxicity risk genotypes” may be selected from one or more of the following rs7853758aa; rs3101826gg; rs3768293 cc; and rs1472408gg.

A “clade” is a group of haplotypes that are closely related phylogenetically. For example, if haplotypes are displayed on a phylogenetic (evolutionary) tree a clade includes all haplotypes contained within the same branch. The pattern of a set of markers along a chromosome is referred to as a “Haplotype”. Accordingly, groups of alleles on the same small chromosomal segment tend to be transmitted together. Haplotypes along a given segment of a chromosome are generally transmitted to progeny together unless there has been a recombination event. In the absence of a recombination event, haplotypes can be treated as alleles at a single highly polymorphic locus for mapping.

As used herein “haplotype” is a set of alleles of closely linked loci on a chromosome that tend to be inherited together. Such allele sets occur in patterns, which are called haplotypes. Accordingly, a specific SNP or other polymorphism allele at one SNP site is often associated with a specific SNP or other polymorphism allele at a nearby second SNP site or other polymorphism site. When this occurs, the two SNPs or other polymorphisms are said to be in Linkage Disequilibrium (LD) because the two SNPs or other polymorphisms are not just randomly associated (i.e. in linkage equilibrium).

In general, the detection of nucleic acids in a sample may depend on the technique of specific nucleic acid hybridization in which the oligonucleotide is annealed under conditions of “high stringency” to nucleic acids in the sample, and the successfully annealed oligonucleotides are subsequently detected (see for example Spiegelman, 1964. Scientific American 210: 48). Hybridization under high stringency conditions primarily depends on the method used for hybridization, the oligonucleotide length, base composition and position of mismatches (if any). High-stringency hybridization is relied upon for the success of numerous techniques routinely performed by molecular biologists, such as high-stringency PCR, DNA sequencing, single strand conformational polymorphism analysis, and in situ hybridization. In contrast to Northern and Southern hybridizations, these aforementioned techniques are often performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

“Oligonucleotides” as used herein are variable length nucleic acids, which may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (Bernfield. and Rottman, 1967. FM. J. Biol. Chem. 242: 4134-43; Sulston et al., 1968. Proc Nat Acad Sci. 60: 409-15; Gillam et al., 1975. Nucleic Acid Res. 2:613-24; Bonora et al., 1990. Nucleic Acid Res. 18: 3155-59; Lashkari et al., 1995. Proc Nat Acad Sci 92: 7912-15; McGall et al., 1996. Proc Nat Acad Sci. 93: 13555-60; Albert et al., 2003. Nucleic Acid Res. 31: e35; Gao et al., 2004. Biopolymers 73: 579-96; and Moorcroft et al., 2005. Nucleic Acid Res. 33: e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

An “addressable collection” as used herein is a combination of nucleic acid molecules or peptide nucleic acids capable of being detected by, for example, the use of hybridization techniques or by any other means of detection known to those of ordinary skill in the art. A DNA microarray would be considered an example of an “addressable collection”.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like SNPs, are often useful in tracking meiotic recombination events as positional markers on chromosomes.

Furthermore, the preferential occurrence of a disease gene in association with specific alleles of linked markers, such as SNPs or other polymorphisms, is called “Linkage Disequilibrium” (LD). This sort of disequilibrium generally implies that most of the disease chromosomes carry the same mutation and the markers being tested are relatively close to the disease gene(s).

For example, in SNP-based association analysis and LD mapping, SNPs can be useful in association studies for identifying polymorphisms, associated with a subject's risk of having a side effect to a drug, such as ototoxicity. Unlike linkage studies, association studies may be conducted within the general population and are not limited to studies performed on related individuals in affected families. In a SNP association study the frequency of a given allele (i.e. SNP allele) is determined in numerous subjects having the side effect of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs may be tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition or side effect of interest. In identifying SNPs associated with ototoxicity, candidate sequences may be selected from those already implicated in the pathway of the condition or disease of interest. Once identified, SNPs found in or associated with such sequences, may then be tested for statistical association with an individual's prognosis or susceptibility to the condition or to the side effect of a medication.

For an LD based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs) and short tandem repeats (STRs). In population genetics linkage disequilibrium refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position (see Akey, J. et al., 2001. Eur J Hum Genet 9: 291-300; and Zhang, K. et al., 2002. Am J Hum Genet. 71: 1386-94). This viewpoint is further substantiated by Khoury et al. (1993. Fundamentals of Genetic Epidemiology. New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for r² is 0.5) are potentially useful in predicting the identity of an allele of interest (i.e. associated with the condition or side effect of interest). A high degree of linkage disequilibrium may be represented by an absolute value for r²>0.7 or by an absolute value for r²>0.8. Additionally, a high degree of linkage disequilibrium may be represented by an absolute value for r²>0.85 or by an absolute value for r²>0.9 or by an absolute value for r²>0.95. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably. LD may be useful for genotype-phenotype association studies. For example, if a specific allele at one SNP site (e.g. “A”) is the cause of a specific clinical outcome (e.g. call this clinical outcome “B”) in a genetic association study then, by mathematical inference, any SNP (e.g. “C”) which is in significant LD with the first SNP, will show some degree of association with the clinical outcome. That is, if A is associated (˜) with B, i.e. A-B and C-A then it follows that C-B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on LD between A and C.

Until the mechanism underlying the genetic contribution to a specific clinical outcome is fully understood, LD helps identify potential candidate causal SNPs and also helps identify a range of SNPs that may be clinically useful for prognosis of clinical outcome or of treatment effect or treatment of side effect. If one SNP within a gene is found to be associated with a specific clinical outcome, then other SNPs in LD will also have some degree of association and therefore some degree of prognostic usefulness.

Polymorphisms in linkage disequilibrium may be identified, for example, using the Haploview program (Barrett. et al., 2005. Bioinformatics 21:263-65 (http://www.broad.mit.edu/mpg/haploview/)) and the LD function in the Genetics Package in R(R Core Development Group, 2005—R Development Core Team (www.R-project.org). Linkage Disequilibrium between markers may be defined using r² whereby all SNPs available on Hapmap.org (phase II) (cohort H), all SNPs genotyped internally using the Illumina Goldengate assay (cohort I) and SNPs may be sequenced using the Sequenom Iplex Platform (cohort S) for genes of interest. A minimum r² of 0.5 may be used as the cutoff to identify LD SNPs.

Numerous sites have been identified as polymorphic sites associated ototoxicity following administration of a pharmacotherapeutic compound having an ototoxicity risk (i.e. cisplatin; see TABLE 1). The polymorphisms in TABLE 1 are linked to (in LD with) numerous polymorphisms as set out in TABLE 3 below, and these LD SNPs may also therefore be indicative of the risk of ototoxicity following platinum-coordinating compound administration. The polymorphisms set out in TABLE 1 relate to the top or forward strand.

TABLE 1
Single nucleotide polymorphisms associated with cisplatin-induced deafness

| | | | | | | | | ADR |
|---|---|---|---|---|---|---|---|---|
| | | | | Odds | | | ADR | Reduced |
| | | SNP | | Ratio | | | Risk | Risk |
| Symbol | SNP ID | Position | Chromosome | (OR) | P Value | SNP | Allele | Allele |
| MTHFR | rs1994798 | 11789021 | 1 | 4.4 | 0.0064 | [A/G] | G | |
| NAT2 | rs2410556 | 18298747 | 8 | 3.6 | 0.00067 | [A/G] | G | |
| SLC28A3 | rs4242626 | 84150265 | 9 | 2.4 | 0.01 | [A/G] | G | |
| SLC28A3 | rs7867504 | 84149790 | 9 | 2.4 | 0.01 | [A/G] | G | |
| SLC28A3 | rs11140511 | 84158459 | 9 | 117 | 0.002 | [A/C] | A | C |
| SLC28A3 | rs4877831 | 84129438 | 9 | 2.0 | 0.05 | [C/G] | G | C |
| SLC28A3 | rs7853758 | 84130480 | 9 | Infinity | 0.005 | [A/G] | G | A |
| TBXAS1 | rs740150 | 139128694 | 7 | 1.7 | 0.01 | [A/G] | G | |
| TBXAS1 | rs6464431 | 139043852 | 7 | 2.8 | 0.03 | [A/T] | A | |
| TPMT | rs12201199 | 18247781 | 6 | 16.98 | 0.000039 | [A/T] | A | |
| TPMT | rs1142345 | 18238897 | 6 | 10.5 | 0.001 | [A/G] | G | |
| TPMT | rs1800460 | 18247207 | 6 | 18.8 | 0.001 | [A/G] | A | |
| COMT | rs4646316 | 18332132 | 2 | 15.0 | 0.00098 | [A/G] | G | A |
| | | | 2 | | | | | |
| COMT | rs9332377 | 18335692 | 2 | 5.4 | 0.001 | [A/G] | A | |
| | | | 2 | | | | | |
| SLC22A1 | rs3101826 | 160504843 | 6 | 5.5 | 0.00096 | [A/G] | A | G |
| XDH | rs207425 | 31403166 | 2 | Infinity | 0.004 | [A/G] | A | |
| EPHA2 | rs3768293 | 16340511 | 1 | 49.2 | 0.000024 | [A/C] | | C |
| EPHA2 | rs1472408 | 16351241 | 1 | 20.6 | 0.0006 | [A/G] | | G |

TABLE 2 shows the flanking sequences for the SNPs shown in TABLE 1 providing their rs designations and corresponding SEQ ID NO designations. Each polymorphism is at position 201 (in bold) within the flanking sequence unless otherwise indicated, and identified in bold. Discrepancies in Table 2 with respect to the polymorphisms indicated in Table 1 (with specific regard to SEQ ID NOs: 1, 2, 3, 4, 6, 8, 10, 13, 14, and 18) reflect reference to the opposite strand. With respect to SEQ ID NOs: 6, 9, and 10, discrepancies may further reflect the difficulty in distinguishing between the two strands since the base pair at the polymorphic site remains identical between polymorphisms.

TABLE 2

| | | | SEQ ID |
|---|---|---|---|
| Symbol | SNP ID | GENOMIC SEQUENCE | NO: |
| MTHFR | rs1994798 | CCTTGTCTCAATTCTCTGTCCCCATCCTCA | 1 |
| | | CCCAGGCGTCCCCTACCCTGGGCTCTCAGC | |
| | | GCCCACCCCAAGCGCCGAGAGGAAGATGTA | |
| | | CGTCCCATCTTCTGGGCCTCCAGACCAAAG | |
| | | AGTTACATCTACCGTACCCAGGAGTGGGAC | |
| | | GAGTTCCCTAACGGCCGCTGGTGAGGGCCT | |
| | | GCAGACCTTCCTTGCAAATAYATCTTTGTT | |
| | | CTTGGGAGCGGGAGGGCAGAAGAAGTTTGC | |
| | | ATGCTTGTGGTTGACCTGGGAGGAGTCAGG | |
| | | GGCAGAATTTACAGGAATGGCCTCCTGGGC | |
| | | ATGTGGTGGCACTGCCCTCTGTCAGGAGTG | |
| | | TGCCCTGACCTCTGGGCACCCCTCTGCCAG | |
| NAT2 | rs2410556 | GCTATCTGTAAAAAAATACGTTTAACATTA | 2 |
| | | AGGTTTATACTTGGGTGAACACCTGATATT | |
| | | CACAGGCTATAAAATAGTTAGCAAGGAAAT | |
| | | AACTTTAAATGTGACTAGTTTTGTCTAATG | |
| | | TCTCAGTTCTCAAAGCAATCTAGGTAAACT | |
| | | GCTAACAATGAATAAATTGAACAAATATAA | |
| | | GTGGGATGGATAAATGCTTGYTGGTTAACT | |
| | | TTTATGTAATTTAAAATCTTAAACTTATTT | |
| | | TGGATTAAAGAACAGCTACTCATTAATAGT | |
| | | TTGGCTCATTTCCAATTAAGTAGAGATATG | |
| | | GAGAAACATGCCTAAAAATTATAGAGTGAT | |
| | | TTCATCTATAAAGTACTGATACCTGATATG | |
| | | CAGTTTAGGATTTCATGTTTCCTAGGTTTA | |
| | | AGGTCACTAAAAATAAAAATTCCACTTAAT | |
| SLC28A3 | rs4242626 | ACATGCTCCTGGGAACAGAGCAGGGGAGAT | 3 |
| | | GGATATCTAACCAAAGCTGAAATAGTCACA | |
| | | GGAAGAGAGTGTTTCTGGCACACTATGAGA | |
| | | TCCTCTGTTAGCTTTTAACCAATGTTTATT | |
| | | ACGTGTTTATTGTTAGAAAATAAAAAGCCA | |
| | | AGGAACTCTGTCATCCCTCTTGGTTTGGCA | |
| | | AAGTTTGAGCAAGTTGGTGGYGCTCTGTCC | |
| | | CCCATCACCATCCCCGTTAGTCCAAAACTG | |
| | | ATGGACCTCATGGGGTGTGCTTAAAATGCA | |
| | | AAGTAGGATTTCCTGGATGTTAGGGCTATT | |
| | | AACCAATGGGTTGTCACAGCTTTCTCAGAA | |
| | | AGCTCTGGAGTTGTTGGAATGTCTTTATTT | |
| | | CCATCCAGGGCTTTGTTATGGGCTGGGTGG | |
| | | GTAGTGTGTGAGTAATGTGTAGGTTGGGTC | |
| SLC28A3 | rs7867504 | CCCCCGATGATTCAGTGAGGTCCCAGAGAA | 4 |
| | | AATAAAGGTTTAATCTTTTTCCACATAAAG | |
| | | TTAATATTTGGGGGAATCCCTGAAAAAAAA | |
| | | AAAAAAAAAGACAGTGGTATCCCTAACCAT | |
| | | CTTTGTTATTTACCTGCTAATAAAATGCCC | |
| | | CAGATGATGTGCCGAAGAGTTGTTTTGTGT | |
| | | TTCCTACAGAAACCACATACYGTGTCATAC | |
| | | CTCCTTTCCAAACACCTGCAACATAAAAGC | |
| | | AAAAAGGCAGGGAGAAGTAAACACCAAAAA | |
| | | CATGAAATAAACTGCATGGTGAGGCTAGAT | |
| | | TAAGCTTTGTAAACTAAGAAGGCAAACAAA | |
| | | CACATGTGTGAGGCGTATGTCTTACAAAAT | |
| | | AGAAATGGGAAACTCATTTCATTTTAGACT | |
| | | AGAGTCAGCCATGATCATGACCATTCAGTG | |
| SLC28A3 | rs11140511 | GGTAGGAGAATTGCTTGAGCCCCAGAGGCA | 5 |
| | | GATCATGCCACTGCACTCCAGCCTGGGAGA | |
| | | CACAGCAAGACTCTACCTGAAAAAAAAAAA | |
| | | AAAGAAAAGAAAAGAAAGTAAAGAAAAGAA | |
| | | ACATATGACTTTTGCTTTGTTTTGTTCTGA | |
| | | TACAAAAGTGGTCCAGGGGAAGAGAGGAAT | |
| | | GAGGACAATTGCCCTCATCTMGGCCTGATA | |
| | | ACTTTCCAATAGGCAGAGCTGGGACCAGGG | |
| | | GCAGGTTTCCACTCTCCTTTCTATCACATC | |
| | | ACACTGATTTTCAACTTATAAAATGAACTA | |
| | | AAACGTGGTAAACCATAGGCTGAATAGCCT | |
| | | TATTCTTAGATGTTAGATTTACGAAATGTT | |
| | | GAAATGGGTTTTTTTTGGTTTAAAGCAATC | |
| | | TCTGGCAGTAA | |
| SLC28A3 | rs4877831 | TTGTCTCAAGGCTTTCAGATCACAGACTCT | 6 |
| | | GAGCCTTTTCTCTCCCTCATCCACGTGGAG | |
| | | AGCAGTGATGATGCTACACTAACTCTCGAG | |
| | | GATGTCCAGGCATGTTACTTTTGGACAGTT | |
| | | TCTCAGAGCTCACAGAATCCATTAAGTTCA | |
| | | AAGGGAGAGTGCTTTAAGATCACTAGACTG | |
| | | TTAGCCACTAGACTATGAGGSCAGGGGCCC | |
| | | CATGACTTGCTATGGCCTCAGTACTCAGCG | |
| | | CATTGCACATAAAAGGCATTCAAAAAATTT | |
| | | TGGTTGATTGATTGCAGTCCCAGCTCATGT | |
| | | GACTCTGGGTCTGTGTCCACAGTCCTCAAA | |
| | | AGTAGATGATTATAGTGGCAATGATTAGCT | |
| | | GTGTCTACACACACACGCACACTCTAATTC | |
| | | AACATAACTGAAAGTTAGGAAGTGCTATGT | |
| SLC28A3 | rs7853758 | GGGATGCATGAGAAGCTTCTACGGTGTGGA | 7 |
| | | AGAGTCTACTGAGGTTAGGGTGGGCTGTTT | |
| | | ACAAACCTATTTTATTTTTAAACAAAGATA | |
| | | GGCAGAAACAAAACAGAGGGCAGGGGCGTG | |
| | | ATGTGATTATACCTCAAAACTCAGCTGTGG | |
| | | GTAGTCAAACATGTTTCCAAACCAGGACAG | |
| | | GGCTGAATTCATAAAAGACARCAGGGCCAG | |
| | | GAAGGCAATCAGATTCACAGCGATGTTGGC | |
| | | CACCAGGGAGATGGAGGAGGATGCTCCCTG | |
| | | TGTTGCAGCTTCTAGAAGATTCCCTGAATC | |
| | | ACTTTATCAAGAAATAGCAATTCCAGAATT | |
| | | ACCAAGGAGTTGTCAGGGGATGGACACCAT | |
| | | TGGTGCAGAAGTAGCATAATCAGAGCTTAG | |
| TBXAS1 | rs740150 | GGGATTTCAGGGCTGCCTGCTGGGGAGGGA | 8 |
| | | GGCTTGAAGCTGTGTCTGCACTGCATCTTC | |
| | | ACAGCACTGAGAAAATGCCAGTCATTCAGG | |
| | | AGGACAGGGCAGCCCTGTCCTCGCCACAGT | |
| | | GCCCGCATCTTCATTGGTTCACCTTTGCAG | |
| | | GTATCATCTGTGACCTGTCATCCAGGCTCT | |
| | | GAGCCCTGAGGATAATAAGTYTCCCTCGAA | |
| | | GGTCCTGGGTTTGTTGGAGCTTTTTCTCCC | |
| | | TCTTGCCATGTCCTTTTCACGGTCCATCCT | |
| | | CTGAGTTTGTGAATTATCTTTCTTCGTGGC | |
| | | CCATGTGGCACAAGATGAAGAGTGCATGGG | |
| | | CAGCTTCTCCCCTGACATCCCTACCTCCCT | |
| | | GACGCTTAACCCACATAGGGGAAGAACATT | |
| | | CCTGAAACAGAGACGTCACCCAAGGTTGCT | |
| TBXAS1 | rs6464431 | GAAATAGTTTACTTCACCATTGTGAATTAT | 9 |
| | | CAACATTACAACCAGTAATTACTAATTACC | |
| | | TCCATTTTGAGTTGATGTTTTGGGGGATGT | |
| | | TTTTATCCTGCAGTGCTATGGAGAGGCTGG | |
| | | GAACGCACTGCTGCCTGTGTTGCTTGTTTA | |
| | | TTTATTTCCTTTTATTCCCAGTGTTCTCTC | |
| | | ATTCCCCTTCCCATCCCCAAWAGTCAACCA | |
| | | TTCCAATTGTGTTTAATGTGTTTTTTTTTC | |
| | | ACATGTTCTTGCAAATTGTGCATTATCATT | |
| | | TCGTGGATATTATCCTTAGTTTATGCCAAT | |
| | | AGTACTGCCGTAGATCTCATTCTATTCCTT | |
| | | AGTTTTTTCATTTAGCACTGTGCTTTCAAG | |
| | | GTCCAGCACAGAGTTCCCATTGAGTCTGCT | |
| TPMT | rs12201199 | TGAAATTCATTTCTATTACAGGCCCAGGTG | 10 |
| | | CAAGGTAGAAGACACTGTCTTACTCACCTT | |
| | | TCTTTTTCCCTAGCTGCCTCAGTTTCCCAT | |
| | | AGTTTGGGAGCTAACCAAAGACAAAACACA | |
| | | TTAAAGTGTGCAGACGAGTGTGTAATAAAA | |
| | | ATATCTGCAGAACAGACATTCAAAAAAATG | |
| | | CTTTGTGGATGTTACACAGGWGGAAGAGAG | |
| | | TGAGGAAGACACCTCCACTCCCATGCCTGC | |
| | | ACTGCCTGGCAAGCATTCAAATTTTTTAAA | |
| | | GTGCAGATGTAGTATTCAACCTACCTGGGA | |
| | | AGATCAAAAATACTGCAACAGTACAATGAA | |
| | | ATGTTCCCCGAAGAACTCTGTAATGAAATA | |
| | | ATGAAAAAAAAATTTTTTTTTTTTACTTAG | |
| TPMT | rs1142345 | CCTGGGAAAGAAGTTTCAGTATCTCCTGTG | 11 |
| | (rs16880254) | TGTTCTTTCTTATGATCCAACTAAACATCC | |
| | | AGGTCCACCATTTTATGTTCCACATGCTGA | |
| | | AATTGAAAGGTTGTTTGGTAAAATATGCAA | |
| | | TATACGTTGTCTTGAGAAGGTTGATGCTTT | |
| | | TGAAGAACGACATAAAAGTTGGGGAATTGA | |
| | | CTGTCTTTTTGAAAAGTTATRTCTACTTAC | |
| | | AGAAAAGTAAATGAGACATAGATAAAATAA | |
| | | AATCACACTGACATGTTTTTGAGGAATTGA | |
| | | AAATTATGCTAAAGCCTGAAAATGTAATGG | |
| | | ATGAATTTTTAAAATTGTTTATAAATCATA | |
| | | TGATAGATCTTTACTAAAAATGGCTTTTTA | |
| | | GTAAAGCCATTTACTTTTTCTAAAAAAGTT | |
| | | TTAGAAGAAAAAGATGTAACTAAACTTTTA | |
| TPMT | rs1800460 | TCCACACCCAGGTCCACACATTCCTCTAGG | 12 |
| | | AGGAAACGCAGACGTGAGATCCTAATACCT | |
| | | TGACGATTGTTGAAGTACCAGCATGCACCA | |
| | | TGGGGGACGCTGCTCATCTTCTTAAAGATT | |
| | | TGATTTTTCTCCCATAAAATGTTTTTTCTC | |
| | | TTTCTGGTAGGACAAATATTGGCAAATTTG | |
| | | ACATGATTTGGGATAGAGGARCATTAGTTG | |
| | | CCATCAATCCAGGTGATCGCAAATGGTAAG | |
| | | TAATTTTTCTTTTTTTGTTTAGCTGTCTTA | |
| | | ATTTTTTAGTATACTATACTTTTTCTGGGT | |
| | | TCTAGAAAATCAGCTTAGACTTCTATGAGT | |
| | | TTGAAATAGGTTATTATGTTTGGAATTTAT | |
| | | AAAAACCTAAATCCAATACTAGCTTTGTCT | |
| COMT | rs4646316 | AGCTCGCTCTGGAGGCACCACCTGAGGTCT | 13 |
| | | GGGAGTGTGGGGGACTGAGGAGGCCCTGTG | |
| | | GTGGGTGGAGATGGGTGGGGAGCTGGGCCA | |
| | | GGGGCCTGGCTGGGTGGCCTGTTGGGAACT | |
| | | GGGGAGCCAGCTGCCTGTGCAGGTGCAAAA | |
| | | TGGGTGGCAGAAGTGGGGTGCACACCCCAG | |
| | | ACCAGACACCAGGGCAGAAAYGGCACAGGA | |
| | | CCAAGGAGATGGGGTGGGGAAGGGCCGCTC | |
| | | TGGGCCCAGCCTGCTCTCCCCCAAGCAAGC | |
| | | CACTGCTCGTGCAAAGAAAGCATGTGTCTC | |
| | | CTGCAGATCTTCCTCCTGAGGCCCCATCTT | |
| | | GTGCATTCCCCCAACCCAGCCCCACTGGCG | |
| | | AGGACCCTGAGTGCCCCGAGTGAGGCTAGA | |
| | | CAGCGGGTGGGGCTGTCCTCGCTTCCCTGG | |
| COMT | rs9332377 | CTGTGTCCTCCCAGGGCCCAGGCACTGGTG | 14 |
| | | AAGATGGGGGGTCTGCAAATGCAGGAGCTT | |
| | | GGGGATGTCCAGAACTGACCCCAAGGGGCA | |
| | | GGCTTGTTGATGGGAGGTCTGCCCCACCTC | |
| | | AGCCCTGCAGGGTCACCCTGGTCAGGCCAA | |
| | | TATTGTCTCCAGGGACCATACCAGCAACCC | |
| | | CTCTCCTTGGGTGCCTCTCCYTCATAGGCC | |
| | | TGAGTTCCTGGCACTGGGTGTTGAGGGCCC | |
| | | CATTGTTTCCACTCACCCAGCTAGCATTTA | |
| | | TTGAGCACCTACTGTGTGCCACATGCTGTT | |
| | | CTAAGGGATGGATACTCCTGAGATGGATAC | |
| | | AGGAGTTGATGAGAGAAAGGTCCCTGTCCT | |
| | | CACGGGGCCCATGTTCTGAAGGTGGCACCC | |
| | | AAGTCTTGTACAGTCCTTTCCTGCAGGAGT | |
| XDH | rs207425 | TATTTTTAGTCTTTTTAATGTTAGTCATTG | 15 |
| | | TGGTGGTTAATGGGTGTGAATTTTAAAACA | |
| | | CATACGCTGTGCACTGCTTAGGATATGTTC | |
| | | ACATGCATTAAAATATAAAAACATGGATGG | |
| | | GAATGTTTTACATCAACCTTAGGATGGTAA | |
| | | TTACCTCAAGGCAGAGGGCAAAATCTCAGA | |
| | | GTGGGGTGATAAAATGGGAARCTTCAACTG | |
| | | TACTTGTAACATTAATCTTTTACCATGGGA | |
| | | AACAAATATGGGCAAACAAACATTTTTTTA | |
| | | GAACCAGGTGGGAAGTATATATGTGTATTT | |
| | | TATCGTTCTCTATATTACTTATGTTTGGTT | |
| | | CATACTGTAATTTTCTTTTTATTTAAGAAA | |
| | | AATGTTTTTAAAGGCAAAAATAGACCC | |
| EPHA2 | rs3768293 | TGGGCTGTGGGGGTTTATGGCCCCTGCCTG | 16 |
| | | GCCTGAGAGCCTGGGCTGGGAACGCCCCCG | |
| | | AGCTTCCCAAGTCAGCTGGCCCTGGACCAC | |
| | | CTGAACCAGCACTGAGCCAGGTGAAGTCTC | |
| | | CTCCACAGACAAGTCAGGGCATTTGGGGGA | |
| | | ACTGACCCCAGGATAAACATGGCCCAGCTT | |
| | | TCTGGAGTCTCAGTTTTACTMACAGATCTG | |
| | | GTGGAGGAGAGAGCTATTTTTGTATCATGC | |
| | | AGATATGTTATGGGGAGGGATGCAATATAC | |
| | | TCATGGGTTTCCAAATACAATAGGAGACTT | |
| | | GAAGACATTCGGGGCTTGAAGGAGACCACT | |
| | | TCAGCCACCCCAACCAACCCCCCTGGGCTG | |
| | | GAGTCTACACTTTTCTGCTCACAGACACAA | |
| | | AAGTGCCCAGTTTGAGAAGATCTGTGGTGG | |
| EPHA2 | rs1472408 | CTGGGGGCCTTCAGCCCAGGTAGGAGGGCC | 17 |
| | | ATGTCACTTCAGGAGGCGGTCTTCAAGACC | |
| | | ACCCTCAGAGCCCAGCTCCCATCTCCACAA | |
| | | ACCAGGCCATCCCTGCTCCCAGCCTGCCTG | |
| | | GAGCTCTGTCCACCCTTTGAGTCCTTCTCC | |
| | | GGTCCTGGCCTTGAGGAATGGGGCTTCTGA | |
| | | GGCAGATCCCTCATGCTCCARGGCCCAAAG | |
| | | GAAGCATTGACTTGGTTTCTTTACCCCCAC | |
| | | CTTAGGGCTTTACCCTCTGAATCCATCTTG | |
| | | CATAGGTTCTATGCCCCGGTTTCTCCTATC | |
| | | TCCTTACCCTCTAGGGAGGGTAGCACTTAT | |
| | | TGGCAGCTACCTGGACTTTACTTGGAAATA | |
| | | GAGTGGGGACAGTACCTAGGGTCTTAGGTT | |
| | | TTGGCTATGGCCTCTGAGCCTGGCAGAGAA | |
| SLC22A1 | rs3101826 | ATCAAGTTCCTCTGGAATCACTCCTATCAG | 18 |
| | | TGGCAAAGCGGTGGACACCCCACGACTATC | |
| | | TCCGCCATTGTCAGGAATTTCAGGAGCTTC | |
| | | ATTGGCTAAGCCTGCAGCACTCAGGGTGAC | |
| | | CCGGCTGGAAGGCAGAGTAGTCGAGAATCA | |
| | | TTCTTTTGGAGACAAGACTGAAAAGGCTTC | |
| | | CCTGGCTGGTCTGAGCAGGAYGTTCTAAGG | |
| | | GTCGCTGCTCCTTGGTGGTGTGAGAAGCAC | |
| | | ATTCTCTTTGGAACTGCAGTAACTAAGCAC | |
| | | CTAGCTGCAACTAGGGCTATGGTGAGTTTG | |
| | | CCTCGATTATTGTTAAATTGCAGTTTACCT | |
| | | GACACCTCACTTGTGATTTAGTTTAAAAAT | |
| | | TTTAAATTACCAAGAAACGGGGGAAAAAAA | |

TABLE 3
Polymorphic sites in linkage disequilibrium (LD) with those listed
in Table 1. A minimum r² of 0.7 was used as the cutoff to
identify LD SNPs. The SNPs identified below were in linkage
disequilibrium with rs1994798; rs2410556; rs4242626; rs7867504;
rs11140511; rs4877831; rs7853758; rs740150; rs6464431;
rs12201199; rs1142345; rs1800460; rs3101826; rs4646316;
rs9332377; rs207425; rs3768293; and rs1472408.

| | | | | ADR- |
|---|---|---|---|---|
| | SNP ID from | SNPs in LD | | Associated |
| Symbol | Table 1 | (r2 > 0.7) | SNP Nucleotides | Variant |
| MTHFR | rs1994798 | rs11121828 | [A/G] | G |
| | | rs12404124 | [T/G] | G |
| | | rs198391 | [T/C] | C |
| | | rs198393 | [C/T] | T |
| | | rs198399 | [T/A] | A |
| | | rs198401 | [A/G] | G |
| | | rs198406 | [A/G] | G |
| | | rs198408 | [T/A] | A |
| | | rs4845882 | [G/A] | A |
| | | rs4846049 | [G/T] | T |
| | | rs4846052 | [C/T] | T |
| | | rs4846054 | [T/G] | G |
| | | rs503040 | [G/A] | A |
| | | rs535107 | [A/G] | G |
| | | rs6541003 | [A/G] | G |
| | | rs6697244 | [T/G] | G |
| | | rs7538516 | [T/C] | C |
| NAT2 | rs2410556 | none r² > 0.7 | | |
| SLC28A3 | rs11140511 | rs7036569 | [T/C] | C |
| | | rs17426961 | [C/T] | T |
| | | rs4585823 | [G/A] | A |
| | | rs17427184 | [G/A] | A |
| | | rs7861242 | [G/A] | A |
| | | rs4877837 | [G/A] | A |
| | | rs7867504 | [A/G] | G |
| | | rs4242626 | [A/G] | G |
| | | rs10868141 | [C/T] | T |
| | | rs10868142 | [C/T] | T |
| | | rs10123041 | [C/T] | T |
| | | rs9792674 | [T/C] | C |
| | | rs4877838 | [C/G] | G |
| | | rs10746739 | [C/T] | T |
| | | rs12005041 | [G/C] | C |
| | | rs7863627 | [C/T] | T |
| | | rs11140511 | [A/C] | A |
| | | rs4877839 | [G/A] | A |
| | | rs4877841 | [G/T] | T |
| | | rs4877842 | [A/G] | G |
| | | rs10780663 | [A/G] | G |
| | | rs7029691 | [G/A] | A |
| | | rs4877844 | [G/C] | C |
| | | rs17336552 | [C/T] | T |
| | | rs10122651 | [A/C] | C |
| SLC28A3 | rs4242626 | rs7867504 | [A/G] | G |
| | | rs4877829 | [A/C] | A |
| | | rs4877832 | [G/A] | G |
| SLC28A3 | rs4877831 | rs7849745 | [C/A] | A |
| | | rs11140481 | [T/C] | C |
| | | rs7857113 | [A/G] | G |
| | | rs7857379 | [A/G] | G |
| | | rs7873208 | [T/C] | C |
| | | rs2184747 | [G/A] | A |
| SLC28A3 | rs7853758 | rs7853066 | [A/G] | A |
| | | rs7047315 | [A/G] | A |
| | | rs10868137 | [A/G] | A |
| | | rs885004 | [A/G] | G |
| | | rs4877836 | [T/C] | T |
| SLC28A3 | rs7867504 | rs11140511 | [A/C] | C |
| TBXAS1 | rs6464431 | rs11973494 | [C/A] | A |
| | | rs6977672 | [T/C] | C |
| TBXAS1 | rs740150 | rs41715 | [A/G] | A |
| | | rs2284211 | [A/G] | G |
| TPMT | rs12201199 | rs12199060 | [T/C] | C |
| | | rs10949481 | [A/T] | T |
| | | rs1142345 | [A/G] | G |
| | | rs6908777 | [G/A] | A |
| | | rs11964408 | [C/T] | T |
| TPMT | rs1800460 | not in | | |
| | | HapMap | | |
| COMT | rs9332377 | rs12485043 | [A/G] | A |
| | | rs9617857 | [G/T] | T |
| | | rs9618725 | [T/C] | C |
| COMT | rs4646316 | rs4646312 | [T/C] | T |
| | | rs740601 | [A/C] | A |
| | | rs2239393 | [A/G] | A |
| | | rs4680 | [A/G] | A |
| SLC22A1 | rs3101826 | rs476235 | [C/T] | C |
| XDH | rs207425 | rs6756897 | [T/C] | C |
| EPHA2 | rs3768293 | rs11260822 | [T/C] | T |
| | | rs12401559 | [C/T] | C |
| | | rs12405694 | [A/G] | A |
| | | rs12408442 | [T/C] | T |
| | | rs12408813 | [G/A] | G |
| | | rs1472408 | [A/G] | A |
| | | rs1566145 | [A/G] | A |
| | | rs2230597 | [C/T] | C |
| | | rs2863841 | [G/C] | G |
| | | rs3820609 | [C/A] | C |
| | | rs6603867 | [G/A] | G |
| | | rs6603883 | [G/A] | G |
| EPHA2 | rs1472408 | rs6678616 | [C/T] | C |
| | | rs3820609 | [C/A] | C |
| | | rs3768293 | [A/C] | A |
| | | rs12405694 | [A/G] | A |
| | | rs1566145 | [A/G] | A |
| | | rs2863841 | [G/C] | G |
| | | rs6603867 | [G/A] | G |
| | | rs6678616 | [C/T] | C |
| | | rs1472408 | [A/G] | A |
| | | rs6603883 | [G/A] | G |
| | | rs11260822 | [T/C] | T |
| | | rs12401559 | [C/T] | C |
| | | rs12408813 | [G/A] | G |
| | | rs12408442 | [T/C] | T |

It will be appreciated by a person of skill in the art that further linked polymorphic sites and combined polymorphic sites may be determined. A haplotype of the above genes can be created by assessing polymorphisms in normal subjects using a program that has an expectation maximization algorithm (for example PHASE). A constructed haplotype of these genes may be used to find combinations of SNPs that are in LD with the tag SNPs (tSNPs) identified herein. Accordingly, the haplotype of an individual could be determined by genotyping other SNPs or other polymorphisms that are in LD with the tSNPs identified herein. Single polymorphic sites or combined polymorphic sites in LD may also be genotyped for assessing subject risk of ototoxicity following platinum-coordinating compound treatment or aminoglycoside compound treatment.

It will be appreciated by a person of skill in the art that the numerical designations of the positions of polymorphisms within a sequence are relative to the specific sequence and the orientation of the strand being read (i.e. forward or reverse). Also the same positions may be assigned different numerical designations depending on the way in which the sequence is numbered and the sequence chosen. Furthermore, sequence variations within the population, such as insertions or deletions, may change the relative position and subsequently the numerical designations of particular nucleotides at and around a polymorphic site. For example, the sequences represented by accession numbers NM_{—}000379, U39487, U06117, D1 1456, CV574002, CR614711, AL709033, AK130114, DQ089481, AL121657, AL121654, AF203979 and AC010743 all comprise XDH nucleotide sequences, but may have some sequence differences and numbering differences between them. Furthermore, one of skill in the art will appreciate that a variety of sequencing, amplification, extension, genotyping or hybridization primers or probes may be designed to specifically identify the polymorphisms described in TABLES 1 and 3, and the sequences flanking the various polymorphisms as provided herein (TABLE 2) are illustrative examples. One of skill in the art will also appreciate that a variety of sequencing, amplification, extension, genotyping or hybridization primers or probes adjacent to, complimentary to, or overlapping with the sequences provided in TABLE 2, may be developed or designed for the identification of the polymorphisms described herein, without going beyond the scope of various embodiments of the invention as described herein. Furthermore, it will be appreciated by a person of skill in the art that the sequences set out herein may be received in either orientation (i.e. forward and reverse) and that the SNP would change accordingly (See, for example, rs1994798 of TABLE 1 as compared to SEQ ID NO: 1 of TABLE 2).

One example of a partial gene sequence is a human XDH gene sequence illustrated as GenBank accession # NM_{—}000379. The genomic sequence of the human XDH gene (NC_{—}000002.10 nucleotides 31410692-31491115) further includes 5′ and 3′ untranslated sequences, introns and the like. Sequence databases with this information, such as GenBank, operated by the National Centre for Biotechnology Information (NCBI) store such information in a retrievable format, and are publicly accessible. A person of skill in the art will appreciate the various methods and tools that may be used to access such information, in a context suitable to their particular application of aspects described herein.

Polymorphic sites in SEQ ID NO: 1-18 are identified by their variant designation (i.e. M, W, Y, S, R, K, V, B, D, H or by “−” for a deletion, a “+” or for example “G” etc. for an insertion).

An “rs” prefix designates a SNP in the database is found at the NCBI SNP database (http://www.ncbi.nlm.nih.gov/entrez/query.fcgi?db=Snp). The “rs” numbers are reference SNP numbers and are in NCBI rsSNP ID form.

The sequences given in TABLE 2 (SEQ ID NO: 1-18) above and those associated with the rs identifiers identified in TABLE 3 may be useful to a person of skill in the art in the design of primers, probes, other oligonucleotides, and/or PNAs for the identification of polymorphisms as described herein.

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the SNP of interest.

A “genotype” is defined as the genetic constitution of an organism, usually in respect to one gene or a few genes or a region of a gene relevant to a particular context (i.e. the genetic loci responsible for a particular phenotype).

A “phenotype” is defined as the observable characters of an organism. In gene association studies, the genetic model at a given locus can change depending on the selection pressures (i.e., the environment), the population studied, or the outcome variable (i.e., the phenotype).

A similar observation would be seen in a gene association study with the hemoglobin, beta gene (HBB) with mortality as the primary outcome variable. A mutation in the HBB gene, which normally produces the beta chain subunit of hemoglobin (B allele), results in an abnormal beta chain called hemoglobin S (S allele; Allison A (1955) Cold Spring Harbor Symp. Quant. Biol. 20:239-255). Hemoglobin S results in abnormal sickle-shaped red blood cells which lead to anemia and other serious complications including death. In the absence of malaria, a gene association study with the HBB gene would suggest a codominant model (survival(BB)>survival (BS)>survival (SS)). However, in the presence of malaria, a gene association study with the HBB gene would suggest a heterozygote advantage model (survival(BB)<survival(BS)>survival(SS)).

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins” or “I”) of a nucleotide relative to a reference allele. Furthermore, a person of skill in the art would appreciate that an insertion or deletion within a given sequence could alter the relative position and therefore the position number of another polymorphism within the sequence. Furthermore, although an insertion or deletion may by some definitions not qualify as a SNP as it may involve the deletion of or insertion of more than a single nucleotide at a given position, as used herein such polymorphisms are also called SNPs as they generally result from an insertion or deletion at a single site within a given sequence.

A “subject”, as used herein, refers to a patient or test subject, for example a human patient, but may also include a mammal. The subject may have been previously diagnosed with a neoplastic disorder, or may be suspected of having a neoplastic disorder and thus may be a candidate for a pharmacotherapeutic regimen. Alternatively, the subject may be a candidate for aminoglycoside therapy. The subject may also be selected as part of a general population (for example a ‘control’ subject), or may be selected as part of a particular ethnic, gender, age or genetic subgroup of a population, or may be excluded from selection as part of a particular ethnic, gender, age or genetic subgroup of a population. Patients and test subjects, whether control or not, may be generally referred to as a subject.

As used herein, the term “approved indication” refers to a symptom or particular circumstance that indicates the advisability or necessity of a specific medical treatment or procedure as sanctioned by a duly authorized regulatory body.

As used herein, the terms “cancer” or “neoplastic condition” or “neoplastic disorder” or “neoplastic disease” refer to a proliferative disorder caused or characterized by the proliferation of cells which have lost susceptibility to normal growth control. A “cancer” or “neoplastic condition” or “neoplastic disorder” or “neoplastic disease” may include tumors and any other proliferative disorders. Cancers of the same tissue type usually originate in the same tissue, and may be divided into different subtypes based on their biological characteristics. Four general categories of cancers are carcinoma (epithelial tissue derived), sarcoma (connective tissue or mesodermal derived), leukemia (blood-forming tissue derived) and lymphoma (lymph tissue derived). Over 200 different types of cancers are known, and every organ and tissue of the body may be affected. Specific examples of cancers that do not limit the definition of cancer may include melanoma, leukemia, astrocytoma, glioblastoma, retinoblastoma, lymphoma, glioma, Hodgkins' lymphoma and chronic lymphocyte leukemia. Examples of organs and tissues that may be affected by various cancers include pancreas, breast, thyroid, ovary, uterus, testis, prostate, thyroid, pituitary gland, adrenal gland, kidney, stomach, esophagus or rectum, head and neck, bone, nervous system, skin, blood, nasopharyngeal tissue, lung, urinary tract, cervix, vagina, exocrine glands and endocrine glands. Alternatively, a cancer may be multicentric or of unknown primary site (CUPS).

As used herein, a “pharmacotherapeutic” refers to a pharmaceutical compound used in the prevention, treatment, or amelioration of a disease or a condition.

As used herein, a “therapeutic regimen” refers to a pharmacotherapeutic regimen or a radiotherapy regimen, or a combination thereof.

As used herein, a “pharmacotherapeutic regimen” or “pharmacotherapy” refers to the use of at least one pharmacotherapeutic compound. Such compounds may be selected from a platinum-coordinating compound or aminoglycoside.

A pharmacotherapeutic compound having an “ototoxicity risk” as used herein refers to any compound used for the treatment, prevention, or amelioration of a disease or condition wherein a potential side effect of the compound is hearing loss. For example, such compounds may be platinum-coordinating compounds or aminoglycosides.

“A platinum-coordination complex” or “platinum-coordination compound” or “platinum-coordinating compound” as used herein is meant to include any tumor cell growth inhibiting platinum-coordinating compound which provides platinum in the form an ion. Platinum-coordinating compounds may, for example, be selected from one or more of the following: cisplatin (trans-diaminedichloro-platinumCII),); cis-diaminedichloroplatinum(II)-ion; cis-diamminediaquoplatinum (I[Iota])-ion; chloro(diethylenetriamine)-platinum(II) chloride; dichloro(ethylenediamine)-platinum(II); carboplatin (diammine(1,1-cyclobutanedicarboxylato)platinum(II)); spiroplatin; iproplatin (dichlorotrans-dihydroxybisisopropolamine platinum IV); diammine(2-ethylmalonato)-platinum(II); ethylenediamine-malonatoplatinum(II); aqua(1,2-diaminodyclohexane)-sulfatoplatinum(II); (1,2-diaminocyclohexane)malonato-platinum(II); (4-caroxyphthalato)(1,2-diaminocyclo-hexane)platinum(II); (1,2-diaminocyclohexane)-(isocitrato)platinum(II); (1,2-diaminocyclohexane)-cis(pyruvato)platinum(II); (1,2-diaminocyclohexane)-oxalatoplatinum(II); oxaliplatin; ormaplatin; tetraplatin; satraplatin; nedaplatin; eptaplatin; lobaplatin. picoplatin; miboplatin; sebriplatin; and aroplatin.

“An aminoglycoside” or “aminoglycoside antibiotic” or “aminoglycoside compound” as used herein refers to any compound useful in the treatment of gram-negative bacteria that can be characterized by amino sugars that have glycosidic linkages including, for example, streptomycin, kanamycin, tobramycin, neomycin, gentamicin, amikacin and netilmicin.

There are a myriad of such pharmacotherapeutic compound available for treating cancer or bacterial infections. Pharmacotherapy agents may be administered to a subject in a single bolus dose, or may be administered in smaller doses over time. A single pharmacotherapeutic compound may be used (single-agent therapy) or more than one agent may be used in combination (combination therapy). Pharmacotherapy may be used alone to treat some types of cancer or some types bacterial infection. Alternatively, pharmacotherapy may be used in combination with other types of treatment, for example, radiotherapy or alternative therapies (for example immunotherapy) as described herein. Additionally, a chemosensitizer may be administered as a combination therapy with a pharmacotherapy agent.

As used herein, a “pharmacotherapeutic compound” or “pharmacotherapy agent” or refers to a medicament. Such medicaments may be used to treat cancer or bacterial infection. In one embodiment, a pharmacotherapeutic generally has the ability to kill cancerous cells directly. Examples of such pharmacotherapeutic compounds include alkylating agents, antimetabolites, natural products, hormones and antagonists, and miscellaneous agents. Examples of alternate names are indicated in brackets. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel and docetaxel (Taxotere); epipodophyllotoxins such as etoposide and teniposide; camptothecins such as topotecan or irinotecan; antibiotics such as dactinomycin (actinomycin D), bleomycin, mitomycin (mitomycin C); anthracycline antibiotics such as daunorubicin (daunomycin, rubidomycin), doxorubicin, idarubicin, epirubicin; enzymes such as L-asparaginase; and biological response modifiers such as interferon alpha and interleukin 2 Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum-coordination complexes such as cisplatin (cis-DDP), carboplatin, oxaliplatin, tetraplatin, ormiplatin, iproplatin or satraplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; or tyrosine kinase inhibitors such as imatinib. Alternate names and trade-names of these and additional examples of pharmacotherapeutic compounds, and their methods of use including dosing and administration regimens, will be known to an individual versed in the art, and may be found in, for example “The Pharmacological basis of therapeutics”, 10th edition. HARDMAN H G., LEvIBIRD L E. editors. McGraw-Hill, New York, or in “Clinical Oncology”, 3rd edition. Churchill Livingstone/Elsevier Press, 2004. ABELOFF, M D. editor.

In another embodiment, a pharmacotherapeutic may generally have the ability to kill bacterial cells. Examples of such pharmacotherapeutic compounds include aminoglycoside antibiotics. Examples of aminoglycoside antibiotics include Gentamicin, Neomycin, Amikacin, Kanamycin, Netilmicin, Streptomycin, and Tobramycin.

The mechanisms underlying these troublesome side effects associated with aminoglycoside antibiotics and platinum-coordinating compounds are reported to involve the production of reactive oxygen species in the cochlea, which can trigger cell-death pathways (Peters et al, 2000. Anticancer Drugs. 11:639-43; Rybak and Whitworth, 2005. Drug Discovery Today. 10: 1313-21; Clerici et al., 1996. Hear Res 98: 116-24). XDH catalyzes the formation of hypoxanthine to xanthine to urate, a major anti-oxidant in blood. XDH deficiency may produce increased sensitivity to free radical induced oxidative stress, which in the ear could be manifested as hearing loss. XDH activity is normally increased in response to cisplatin administration, possibly as a protective response to the formation of free radicals (Kizilay et al., 2004. J Chemother 16, 381-87; Sogut et al., 2004. Cell Biochem Funct 22, 157-62).

Cisplatin normally binds thiol-containing compounds and purines, especially guanine, and exerts its cytotoxic effect by forming intra-strand and inter-strand DNA cross-links, causing cell death in rapidly dividing cells. TPMT can methylate and inactivate exogenous thiopurine compounds, such as the metabolites of azathioprine (Weinshilboum et al., 2006. Cell Mol Neurobiol 26: 539-61; Weinshilboum et al., 1980. Am J Hum Genet. 32: 651-62). It is possible that a loss of TPMT enzyme activity could also reduce the inactivation of cisplatin-purine compounds, thereby increasing the efficiency of cisplatin cross-linking, and increasing cisplatin toxicity.

S-adenosyl methionine (SAM) substantially increases cisplatin-induced toxicity in cisplatin-treated mice (Ochoa et al., 2009. Arch Med Res. 40: 54-85). Administration of SAM alone is not toxic, and administration of cisplatin alone exhibits moderate toxicity, while administration of SAM and cisplatin dramatically increase cisplatin toxicity, as monitored by renal dysfunction (creatinine and BUN). These results suggest that cisplatin-induced ototoxicity could be related to increased levels of SAM. TPMT and COMT are methyltransferases dependent on SAM methyl donor substrate in the methionine pathway (Weinshilboum et al., 2006. Cell Mol Neurobiol 26: 539-61; Weinshilboum et al., 1980. Am J Hum Genet. 32: 651-62). COMT-like enzyme activity is involved in auditory function in mice and humans (Ahmed et al., 2008. Nat Genet. 40: 1335-40; Du et al., 2008. Proc Natl Acad Sci USA. 105: 14609-14).

One strategy to protect the inner ear from ototoxicity is the administration of antioxidant drugs to provide upstream protection and block the activation of cell-death sequences. Downstream prevention involves the interruption of the cell-death cascade that has already been activated, to prevent apoptosis. Challenges and opportunities exist for appropriate drug delivery to the inner ear and for avoiding interference with the therapeutic efficacy of both categories of ototoxic drugs.

Once a subject is identified as a candidate for administration of a pharmacotherapeutic having an ototoxicity risk, then genetic sequence information may be obtained from the subject to assess the risk of ototoxicity for the subject. Or alternatively, genetic sequence information may already have been obtained from the subject to determine ototoxicity risk or to identify the subject's genotype prior to becoming a candidate for administration of a pharmacotherapeutic having an ototoxicity risk. For example, a subject may have already provided a biological sample for other purposes or may have even had their genetic sequence determined in whole or in part and stored for future use. Genetic sequence information may be obtained in numerous different ways and may involve the collection of a biological sample that contains genetic material, particularly, genetic material containing the sequence or sequences of interest. Many methods are known in the art for collecting biological samples and extracting genetic material from those samples. Genetic material can be extracted from blood, tissue, hair and other biological material. There are many methods known to isolate DNA and RNA from biological material. Typically, DNA may be isolated from a biological sample when first the sample is lysed and then the DNA is separated from the lysate according to any one of a variety of multi-step protocols, which can take varying lengths of time. DNA isolation methods may involve the use of phenol (Sambrook, J. et al, “Molecular Cloning”, Vol. 2, pp. 9.14-9.23, Cold Spring Harbor Laboratory Press (1989) and Ausubel, Frederick M. et al, “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The resulting nucleic acid sample may be used ‘as-is’ in further analyses or may be purified further. Additional purification of the precipitated, crude DNA fraction may be achieved by any one of several methods, including, for example, column chromatography (Analects, (1994) VoI 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves Iy sing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

The level of expression of specific nucleic acids such as mRNAs or microRNAs, copy number of a gene, or the degree of heterozygosity for a polymorphism may also be determined once the nucleic acid sample has been obtained. Quantitative and semi-quantitative methods are known in the art, and may be found in, for example AUSUBEL, supra; SAMBROOK, supra or Harrison's Principles of Internal Medicine 15th ed. BRAUNWALD et al eds. McGraw-Hill.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR),

Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of apolymorphism in an ototoxicity associated gene sequence, as described herein.

Detection or determination of a nucleotide identity, or the presence of one or more single nucleotide polymorphism(s) (SNP typing), may be accomplished by any one of a number methods or assays known in the art. Many DNA typing methodologies are useful for use in the detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids.

Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length. A unimolecular segment amplification method for amplifying nucleic acids is described in U.S. Pat. No. 5,854,033. A rolling circle replication reporter system may be used for identification of polymorphisms or mutations.

An invasive cleavage method employs an “Invader™ (Applied Biosystems) probe and sequence-specific probes to hybridize with the target nucleic acid, usually DNA, with an overlap of one nucleotide. When the sequence specific probe is an exact match to the site of polymorphism, the overlapping probes form a structure that is specifically cleaved by a FLAP endonuclease, Release of the 5′ end of the allele-specific probe may be detected by known methods as described. See for example, Lu, M., et al. J. Am. Chem. Soc. 2001, 124, 7924-7931; Lyamichev, et al. 1999. Nature Biotech. 17, 292-296; Landegren et al. 1998. Genome Research, 8, 769-776; Brookes, 1999. Gene 234, 177-186; Chen, et al 2004. J. Am. Chem. Soc. 126, 3016-3017; Wang, D. G., et al. Science 1998, 280, 1077-1082. The TaqMan™ assay (Applied Biosystems) exploits the 5′ exonuclease activity of the Taq polymerase to displace and cleave an oligonucleotide probe hybridized to the target nucleic acid, usually DNA, generating a fluorescent signal. See, for example U.S. Pat. Nos. 4,683,202, 4,683,195, and 4,965,188.

5′ exonuclease activity or TaqMan™assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

The Illumina Golden Gate™Assay uses a combined oligonucleotide ligation assay/allele-specific hybridization approach (SHEN R et al Mutat Res 2005573: 70-82). The first series of steps involve the hybridization of three oligonucleotides to a set of specific target SNPs; two of these are fluorescently-labelled allele-specific oligonucleotides (ASOs) and the third a locus-specific oligonucleotide (LSO) binding 1-20 bp downstream of the ASOs. A second series of steps involve the use of a stringent polymerase with high 3′ specificity that extends only oligonucleotides specifically matching an allele at a target SNP. The polymerase extends until it reaches the LSO. Locus-specificity is ensured by requiring the hybridization of both the ASO and LSO in order that extension can proceed. After PCR amplification with universal primers, these allele-specific oligonucleotide extension products are hybridized to an array which has multiple discretely tagged addresses (in this case 1536 addresses) which match an address embedded in each LSO. Fluorescent signals produced by each hybridization product are detected by a bead array reader from which genotypes at each SNP locus may be ascertained.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP system.

Polymorphism detection methods may include but are not limited to the following:

Restriction Fragment Length Polymorphism (RFLP) strategy—An RFLP gel-based analysis can be used to indicate the presence or absence of a specific mutation at polymorphic sites within a gene. Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. PCR primers may be designed using ExonPrimer software and may be synthesized by Invitrogen (USA). PCR reaction products may be purified using Qiaquik 96 Purification Kit (Qiagen, Canada). Finally, if both the higher molecular weight band and the two lower molecular weight bands are visible then the DNA sample contained both polymorphisms, and therefore the DNA sample, and by extension the subject providing the DNA sample, was heterozygous for this polymorphism; For example the Maxam-Gilbert technique for sequencing (MAXAM A M. and GILBERT W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et at. (Proc. Natl. Acad. Sci. USA (1977) 74(12): 5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Q[beta] replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4): 1760-1764). Other methods include those of Donis-Keller et at. (1977, Nucl. Acids Res. 4:2527-2538), SMONCSITS A. et at. (Nature (1977) 269(5631):833-836), AXELROD V D. et at. (Nucl. Acids Res. (1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products. DNA may be sequenced, for example, using fluorescent dye-terminator chemistry on the ABI PRISM® 3100 Genetic Analyzer (Applied Biosystems). Sequencing primers may be designed using ExonPrimer software and may be synthesized by Invitrogen (USA). Sequence data may be analyzed using the Phred/Phrap/Consed software package (Genome Software Development, University of Washington (Seattle, Wash., USA);
Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.
A template-directed dye-terminator incorporation with fluorescent polarization-detection (TDI-FP) method is described by FREEMAN B D. et al. (J MoI Diagnostics (2002) 4(4):209-215) for large scale screening;
Oligonucleotide ligation assay (OLA) is based on ligation of probe and detector oligonucleotides annealed to a polymerase chain reaction amplicon strand with detection by an enzyme immunoassay (VrLLAHERMOSA M L. J Hum Virol (2001) 4(5):238-48; ROMPPANEN E L. Scand J Clin Lab Invest (2001) 61(2):123-9; IANNONE M A. et al. Cytometry (2000) 39(2): 131-40);
Ligation-Rolling Circle Amplification (L-RCA) has also been successfully used for genotyping single nucleotide polymorphisms as described in QI X. et al. Nucleic Acids Res (2001) 29(22):E116;
5′ nuclease assay has also been successfully used for genotyping single nucleotide polymorphisms (AYDIN A. et al. Biotechniques (2001) (4):920-2, 924, 926-8.);
Polymerase proofreading methods are used to determine SNPs identities, as described in WO 0181631;
Detection of single base pair DNA mutations by enzyme-amplified electronic transduction is described in PATOLSKY F et al. Nat. Biotech. (2001) 19(3):253-257;
Gene chip or microarray technologies are also known for single nucleotide polymorphism discrimination whereby numerous polymorphisms may be tested for simultaneously on a single array (for example: EP 1120646; and GILLES P N. et al. Nat. Biotechnology (1999) 17(4):365-70); Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43: 1151-1158; LITTLE D P. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

U.S. Pat. No. 7,074,597 describes methods for multiplex genotyping using solid phase capturable dideoxynucleotides and mass spectrometry. Nucleotide identity is detected at a specific site of a nucleic acid sample by contacting DNA-primer complex with labeled dideoxynucleotides (ddNTPs) to generate labeled single base extended (SBE) primer. The identifying ddNTP may be within the SBE primer.

Multiplex analysis of PCR-amplified products may also be used to detect specific SNPs. Reporting DNA sequences comprising a fluorophore on a 5′ end may be used to combine a multiplex PCR amplification reaction with microsphere based hybridization (U.S. Pat. No. 7,083,951). Other multiplex detection methods include BeadArray™ and similar hybridization-based methods, for example, those described in U.S. Pat. Nos. 6,429,027, 6,396,995, 6,355,431.

Microarray or ‘gene chips’ of oligonucleotides may be used for SNP discrimination. Oligonucleotides may be nucleic acids or modified nucleic acids, including PNAs, and may be ‘spotted’ onto a solid matrix, such as a glass or plastic slide. Alternatively, oligonucleotides may be synthesized in situ on the slide. See, for example, GAO et al 2004. Biopolymers 73:579-596; U.S. Pat. No. 5,445,934; U.S. Pat. No. 5,744,305, U.S. Pat. No. 5,800,992, U.S. Pat. No. 5,796,715.

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites. If a risk is found, a decision may be made as to alternative treatments, adjunct therapies to reduce ototoxicity risk, and/or subject monitoring.

Once the identity of a polymorphism(s) is determined an indication may be obtained as to the subject's risk of ototoxicity following administration of a pharmacotherapeutic compound having an ototoxicity risk. Methods for predicting a subject's risk of ototoxicity following administration a pharmacotherapeutic compound having an ototoxicity risk may be useful in making decisions regarding the selection of a therapeutic regimen comprising one or more pharmacotherapeutic compounds having an ototoxicity risk or the administration of a pharmacotherapeutic compound having an ototoxicity risk.

For example, a subject may be tested for a risk polymorphism before undergoing a therapeutic regimen involving a pharmacotherapeutic compound having an ototoxicity risk. If a subject's genotype included a decreased risk polymorphism or decreased risk allele, this may indicate that the subject is at a low risk for ototoxicity. The identification of one or more decreased risk alleles may thus indicate the relative safety of treating the subject with the pharmacotherapeutic having an ototoxicity risk or the safety of administering an increased dose of pharmacotherapeutic having an ototoxicity risk.

Conversely, if a subject's genotype includes an ototoxicity-associated risk polymorphism or risk allele, this may indicate that the subject is at a risk for ototoxicity. The identification of one or more risk alleles may indicate a need to administer the pharmacotherapeutic having an ototoxicity risk at a lower dosage; eliminate the dose of a pharmacotherapeutic compound having an ototoxicity risk, substitute the pharmacotherapeutic compound having an ototoxicity risk with an alternative therapeutic having no ototoxicity risk or a reduced ototoxicity risk, and/or concomitantly administer an adjunct therapy to reduce the risk of ototoxicity.

Alternative therapeutics having no ototoxicity risk or a reduced ototoxicity risk may include alternative formulations of the pharmacotherapeutic having an ototoxicity risk or alternative pharmacotherapeutics. Alternative formulations of the pharmacotherapeutic having an ototoxicity risk which have a reduced ototoxicity risk may include liposomal formulations that target specific tissues and to reduce the overall toxic effects on normal tissue. Examples of liposomal formulations of pharmacotherapeutics having an ototoxicity risk include SLIT-Cisplatin, Lipoplatin, LiPloxa, MBP324, Lipisomal Carboplatin, and Aroplatin.

Alternative pharmacotherapeutic compounds having no ototoxicity risk or a reduced ototoxicity risk may include, for example, oxaliplatin (Hellberg et al., (2009). J Natl Cancer Inst. 101: 37-47), carboplatin (Watanabe et al., 2002. Chemotherapy 48: 82-87), etoposide, vincristine, paclitaxel, docetaxel, 5-FU, vinblastine, doxorubicin, cyclophosphamide, bleomycin, actinomycin D, methotrexate, tamoxifen, hexamethylmelamine, vinorelbine, ifosfamide and the like.

Examples of adjunct therapies to reduce risk of ototoxicity may include the otoprotectants listed in Table 4 and Table 5, xanthine dehydrogenase inhibitors such as allopurinol, and

Fosfomycin. Subjects may be routinely monitored for signs of ototoxicity as described herein, and the therapeutic regimen revised or adjusted accordingly.

TABLE 4
Effects of protective agents against aminoglycoside ototoxicity

| Aminoglycoside | Protective agent | Species | Efficacy^{a} |
|---|---|---|---|
| Gentamicin | 2,2-DPD or cyclosporine A | Guinea pig | ++ |
| Gentamicin | G protein inhibitor | Rat | +++ |
| | (GDP-βs) | | |
| Gentamicin | Ras inhibitor (FTI277) | Rat | +++ |
| Gentamicin | Ras inhibitor (B581) | Rat | ++ |
| Gentamicin | C. difficile toxin | Rat | ++ |
| Neomycin | D-JNKI-1 | Mouse | +++ |
| Neomycin | D-JNKI-1 | Guinea pig | +++ |
| Gentamicin | α-Tocopherol | Guinea pig | +++ |
| Gentamicin | D-methionine | Guinea pig | ++ |
| Amikacin | Lipoic acid | Guinea pig | +++ |
| Gentamicin | Ebselen | Guinea pig | +++ |
| Gentamicin | Salicylate | Guinea pig | +++ |
| Gentamicin | Ginkgo biloba extract | Guinea pig | +++ |
| Gentamicin | Gu Siu Bu | Guinea pig | ++ |
| Gentamicin | Danshen | Mouse | ++ |
| Gentamicin | Danshen | Mouse | +++ |
| Kanamycin + | Dexamethasone | Guinea pig | + |
| Ethacrynic acid | | | |
| Gentamicin | Dexamethasone + liver | Chinchilla | +++ |
| | extract | | |
| Kanamycin + | GDNF + TGF-β1 | Guinea pig | ++ |
| Ethacrynic acid | | | |
| Gentamicin | SOD analog (M40403) | Mouse | ++ |
| Kanamycin + | SOD1 or SOD2 | Guinea pig | ++ |
| Ethacrynic acid | | | |
| Amikacin | Amakacin preconditioning | Guinea pig | ++ |
| Gentamicin | Ethacrynic acid | Guinea pig | + |
| Gentamicin | CEP1347 | Guinea pig | + |
| Gentamicin | Minocycline | Rat | ++ |
| Gentamicin | Minocycline or p38 MAPK | Rat | +++ |
| | Inhibitor (SB203580) + | | |
| | caspase 3 Inhibitor | | |
| | (DEVD or ZVAD) | | |

Table reproduced from Rybak and Whitworth, 2005. Drug Discovery Today. 10: 1313-21.
^{a}Key: +, low efficacy; ++, moderate efficacy; +++, high efficacy.

TABLE 5
Effects of protective agents against cisplatin ototoxicity

| | | Degree of |
|---|---|---|
| Agent | Species | protection^{a} |
| Thiosulfate | Guinea pig | +++ |
| Thiosulfate | Guinea pig | 0 |
| Amifostine | Hamster | +++ |
| Glutathione ester | Rat | + |
| Diethyldithiocarbamate | Rat | ++ |
| Methylthiobenzoic acid | Rat | ++ |
| Ebselen | Rat | +++ |
| Ebselen & Allopurinol | Rat | ++ |
| Salicylate | Rat | ++ |
| Salicylate | Rat | +++ |
| α-Tocopherol | Rat | ++ |
| α-Tocopherol | Guinea pig | ++ |
| Trolox | Guinea pig | ++ |
| α-Tocopherol + tiopronin | Guinea pig | ++ |
| Tiopronin | Rat | ++ |
| Aminoguanidine | Rat | ++ |
| R-PIA | Chinchilla | ++ |
| CCPA | | ++ |
| Z-DEVD-fluoromethyl | Guinea pig | +++ |
| ketone | | |
| (caspase-3 inhibitor) | | |
| Z-LEKD-fluoromethyl | Guinea pig | +++ |
| ketone | | |
| (caspase-9 inhibitor) | | |
| Pifithrin | | ++ |
| D-JNKI 1 | Guinea pig | 0 |
| M40403 | | 0 |
| D-methionine | Rat | +++ |

Table reproduced from Rybak and Whitworth, 2005. Drug Discovery Today. 10: 1313-21.
^{a}Key: +, low efficacy; ++, moderate efficacy; +++, high efficacy.

### Treatment

Pharmacotherapeutic compounds having an ototoxicity risk, for example, platinum-coordinating compounds or aminoglycosides, are used to treat a variety of bacterial infections and cancers in children and adults. In a given therapeutic regimen, the pharmacotherapeutic having an ototoxicity risk may be administered alone or in combination with other therapeutic agents in various doses and compositions, depending on the approved indication, age of subject, health of subject, body mass, etc. The choice of dose, pharmacotherapeutic compounds or combinations, methods of administration and the like will be known to those skilled in the art. Further, methods of assessing response to treatment and side effects are also known. For example, hearing loss in a subject suspected of experiencing ototoxicity may be assessed by various methods used in audiological assessment, including medical history, conduction testing, speech audiometry, or other methods that may be dependent on the age and condition of the subject, as are known in the art. For example, the use of Brock's criteria (BROCK et al 1991. Med Pediatr Oncol 19:295-300) for scoring the high-frequency hearing loss associated with platinum-coordinating compounds in children may be particularly suitable.

Response to a therapeutic regimen may be monitored. Tumor staging provides a method to assess the size and spread of a tumor in response to a treatment regimen. The TNM tumor staging system uses three components to express the anatomic extent of disease: T is a measure of the local extent of tumor spread (size), N indicates the presence or absence of metastatic spread to regional lymph nodes, and M specifies the presence or absence of metastatic spread to distant sites. The combination of these classifications combine to provide a stage grouping. Clinical TNM (cTNM) defines the tumor based on clinical evidence. Pathologic TNM (pTNM) defines the tumor based on examination of a surgically resected specimen.

Changes in tumor size may be observed by various imaging methods known to physicians or surgeons in the field of oncology therapy and diagnostics. Examples of imaging methods include positron emission tomography (PET) scanning, computed tomography (CT) scanning, PET/CT scanning, magnetic resonance imaging (MRI), chemical shift imaging, radiography, bone-scan, mammography, fiberoptic colonoscopy or ultrasound. Contrast agents, tracers and other specialized techniques may also be employed to image specific types of cancers, or for particular organs or tissues, and will be known to those skilled in the art. Changes in rate of metastasis may also be observed by the various imaging methods, considering particularly the appearance, or frequency of appearance, of tumors distal to the primary site. Alternatively, the presence of tumor cells in lymph nodes adjacent and distal to the primary tumor site may also be detected and used to monitor metastasis.

A subject may be tested for a risk polymorphism before undergoing a therapeutic regimen involving a pharmacotherapeutic compound having an ototoxicity risk. If a subject's genotype includes an ototoxicity-associated polymorphism or risk polymorphism, this may indicate that the subject is at a risk for ototoxicity.

Alternatively, a subject at risk for ototoxicity may be administered a therapeutic regimen of the pharmacotherapeutic compound having an ototoxicity risk and have their hearing acuity monitored as described. If a decrease in hearing acuity is identified, the therapeutic regimen may be altered to decrease the dose of the pharmacotherapeutic compound having an ototoxicity risk, eliminate the dose pharmacotherapeutic compound having an ototoxicity risk, increase the dose of a second regimen having a reduced risk or no risk, administering a pharmacotherapeutic compound having a reduced ototoxicity risk or no ototoxicity risk, or administering an adjunct therapy to reduce the risk of ototoxicity. Examples of platinum-coordinating compounds with reduced ototoxicity risk may include oxaliplatin (Hellberg et al., (2009). J Natl Cancer Inst. 101: 37-47) and carboplatin (Watanabe et al., 2002. Chemotherapy 48: 82-87). Examples of pharmacotherapeutic compounds that may be used in combination with a platinum-coordinating compound in a therapeutic regimen may include, for example, etoposide, vincristine, paclitaxel, docetaxel, 5-FU, vinblastine, doxorubicin, cyclophosphamide, bleomycin, actinomycin D, methotrexate, tamoxifen, hexamethylmelamine, vinorelbine, ifosfamide and the like. Alternatives to aminoglycoside pharmacotherapeutics include ampicillin, chloramphenicol, and nalidixic acid.

For example, the therapeutic regimen may be supplemented to include a xanthine dehydrogenase inhibitor. Examples of xanthine dehydrogenase inhibitors include allopurinol. Alternatively, Fosfomycin is also known to attenuate ototoxicity of platinum-containing anti-tumor agents and may be administered in conjunction with a platinum-coordinating compound.

### Genes

Numerous genes are known to be involved in ADME (absorption, distribution, metabolism and elimination), for example MTHFR, NAT2, SLC28A3, SLC22A1, TBXAS1, TPMT, COMT, XDH, and EPHA2. Detailed information relating to the sequence, expression patterns, molecular biology, etc of these and related genes in both Homo sapiens and in other model species is known, and may be found at, for example Entrez Gene (http://www.ncbi.nlm.nih.gov) and references therein.

5,10-methylenetetrahydrofolate reductase (NADPH) [Homo sapiens] (MTHFR) (alternate names include Methylenetetrahydrofolate reductase; methylenetetrahydrofolate reductase intermediate form) maps to chromosome 1p36.3. The genomic region (chromosome) can be accessed in the NCBI Entrez Genome database by accession number NC_{—}000001, about nucleotides (complement) 11768374-11788702 (in version NC_{—}000001.9, GI:89161185, genome annotation build 36 version 3). Examples of nucleic acid sequences comprising MTHFR include those found in the NCBI Entrez Gene database by accession number NM_{—}005957 (gene ID 4524), and the Ensemb1 database by gene ID ENSG00000177000. MTHFR catalyzes the conversion of 5,10-methylenetetrahydrofolate to 5-methyltetrahydrofolate (THF).

N-acetyltransferase 2 [Homo sapiens] (NAT2) (alternate names include arylamine N-acetyltransferase; arylamide acetylase 2; arylamine N-acetyltransferase 2; AAC2) maps to chromosome 8p22. The genomic region (chromosome) can be accessed in the NCBI Entrez Genome database by accession number NC_{—}000008, about nucleotides 18293035-18303003 (in version NC_{—}000008.9, GI:51511724 genome annotation build 36 version 3). Examples of nucleic acid sequences comprising NAT2 include those found in the NCBI Entrez Gene database by accession number NM_{—}000015 (gene ID 10), and the Ensemb1 database by gene ID ENSG00000156006. NAT2 acetylation functions to both activate and deactivate arylamine and hydrazine drugs and carcinogens.

Solute carrier family 28 (sodium-coupled nucleoside transporter), member 3′ [Homo sapiens] (SLC28A3) (alternate names include concentrative Na+-nucleoside cotransporter; concentrative nucleoside transporter 3; CNT3) maps to chromosome 9q22.2. The genomic region (chromosome) can be accessed in the NCBI Entrez Genome database by accession number NC_{—}000009, about nucleotides (complement) 86082912-86173233 (in version NC_{—}000009.10 GI:89161216, genome annotation build 36 version 3). Examples of nucleic acid sequences comprising SLC28A3 include those found in the NCBI Entrez Gene database by accession number NM 022127 (gene ID 64078), and the Ensemb1 database by gene ID ENSG00000197506. SLC28A3 shows broad specificity for pyrimidine and purine nucleosides. Nucleoside transporters, such as SLC28A3, regulate multiple cellular processes, including neurotransmission, vascular tone, adenosine concentration in the vicinity of cell surface receptors, and transport and metabolism of nucleoside drugs.

Solute carrier family 28 (sodium-coupled nucleoside transporter), member 1′ [Homo sapiens] (SLC28A1) (alternate names include human Organic Cation Transporter 1; hOCT1) maps to chromosome 6q26. The genomic region (chromosome) can be accessed in the NCBI Entrez Genome database by accession number NC_{—}000006.10, about nucleotides (complement) 160462853-160499740. Examples of nucleic acid sequences comprising SLC28A1 include those found in the NCBI Entrez Gene database by accession number U77086 (gene ID 6580), and the Ensemb1 database by gene ID ENSG00000175003. SLC28A1 is one of three similar cation transporter genes located in a cluster on chromosome 6. Polyspecific organic cation transporters in the liver, kidney, intestine, and other organs are critical for elimination of many endogenous small organic cations as well as a wide array of drugs and environmental toxins. The encoded SLC28A1 protein contains twelve putative transmembrane domains and is a plasma integral membrane protein. Two transcript variants encoding two different isoforms have been found for this gene, but only the longer variant encodes a functional transporter.

Thromboxane A synthase 1 [Homo sapiens] (TBXAS1) (alternate names include thromboxane A synthase 1 (platelet, cytochrome P450, family 5, subfamily A); TXA synthase; thromboxane A synthase 1 (platelet, cytochrome P450, subfamily V); cytochrome p450 subfamily V; TS; TXS; CYP5; THAS; TXAS; CYP5A1; GHOSAL) maps to chromosome 7q34-35. The genomic region (chromosome) can be accessed in the NCBI Entrez Genome database by accession number NC_{—}000007, about nucleotides 139175421-139366471 in version NC_{—}000007.12, GI:89161213, genome annotation build 36 version 3). Examples of nucleic acid sequences comprising TBXAS1 include those found in the NCBI Entrez Gene database by accession number NM_{—}001061 or NM_{—}030984 (gene ID 6916), and the Ensemb1 database by gene ID ENSG00000059377. TBXAS 1 catalyzes the conversion of the prostaglandin endoperoxide (H2) into thromboxane A2, a potent vasoconstrictor and inducer of platelet aggregation. TBXAS1 is a endoplasmic reticulum membrane protein and a member of the cytochrome P450 superfamily of enzymes.

Thiopurine s-methyltransferase [Homo sapiens] (TPMT) (alternate names include thiopurine s-methyltransferase; S-adenosyl-L-methionine:thiopurine S-methyltransferase) maps to chromosome 6p22.3. The genomic region (chromosome) can be accessed in the NCBI Entrez Genome database by accession number NC_{—}000006, about nucleotides (complement) 18236521-18263353 in version NC_{—}000006.10, GI:89161210, genome annotation build 36 version 3). Examples of nucleic acid sequences comprising TPMT include those found in the NCBI Entrez Gene database by accession number NM_{—}000367 (gene ID 7172), and the Ensemb1 database by gene ID ENSG00000137364.TPMT is an enzyme that metabolizes thiopurine drugs via S-adenosyl-L-methionine as the S-methyl donor and S-adenosyl-L-homocysteine as a byproduct.

Catechol O-methyltransferase (COMT) maps to chromosome 22q11.21. The genomic region (chromosome) can be accessed in the NCBI Entrez Genome database by accession number NC_{—}000022.9, about nucleotides (complement) 18309309-18336530. Examples of nucleic acid sequences comprising COMT include those found in the NCBI Entrez Gene database by accession number NM_{—}000754 (gene ID 1312), and the Ensemb1 database by gene ID ENSG00000093010. COMT is involved in the inactivation of the catecholamine neurotransmitters (dopamine, epinephrine, and norepinephrine). The enzyme introduces a methyl group to the catecholamine, which is donated by S-adenosyl methionine. COMT is an intracellular enzyme located in the postsynaptic neuron.

Ephrin receptor A2 (EPHA2) maps to chromosome 1p36. The genomic region (chromosome) can be accessed in the NCBI Entrez Genome database by accession number NC_{—}000001.9, about nucleotides (complement) 16323419-16355151. Examples of nucleic acid sequences comprising EPHA2 include those found in the NCBI Entrez Gene database by accession number NM_{—}004431 (gene ID 1969), and the Ensemb1 database by gene ID ENSG00000142627. EPHA2 belongs to the ephrin receptor subfamily of the protein-tyrosine kinase family. EPH and EPH-related receptors have been implicated in mediating developmental events, particularly in the nervous system. Receptors in the EPH subfamily typically have a single kinase domain and an extracellular region containing a Cys-rich domain and 2 fibronectin type III repeats. The ephrin receptors are divided into 2 groups based on the similarity of their extracellular domain sequences and their affinities for binding ephrin-A and ephrin-B ligands. This gene encodes a protein that binds ephrin-A ligands.

Xanthine dehydrogenase [Homo sapiens] (XDH) (alternate names and abbreviations include XO; XOR; xanthene dehydrogenase; xanthine oxidase; xanthine oxidoreductase) maps to chromosome 2p23.1 (about nucleotides 31410692-314911 15 of Build 36.1). Examples of nucleic acid sequences comprising XDH include those found in GenBank under accession numbers NM_{—}000379.3, DQ089481, chromosome 2 NC_{—}000002 (nt 31410692-31491115), U06117, U39487. The XDH gene contains 36 exons and spans at least 60 kb. The exon sizes range from 53 to 279 bp, and the intron sizes range from 0.2 to more than 8 kb. XDH is involved in the oxidative metabolism of purines, and is active as a homodimer.

### Methods

### Patient Recruitment and Sample Collection

Study participants were recruited by the Canadian Pharmacogenomics Network for Drug Safety (CPNDS), a national multi-centre active surveillance consortium for studying adverse drug reactions in children.

Biological samples (blood, saliva, buccal swabs) were collected from two groups of patients: (1) adverse drug reaction (ADR) patients, who experienced a serious or life-threatening ADR that are identified by the hospital-based pharmacists; and (2) drug-matched control patients who receive the target drug but do not experience an ADR that are recruited by clinical pharmacists. When feasible, samples are collected from parents of ADR patients at the same time as the ADR patients.

For each identified ADR case, the clinicians completed an electronic ADR report, provided patients/guardians with information about the study, and obtained patient/parent consent for sample and data collection. Control patients were recruited by the clinicians using the same method as outlined for ADR patients, using the same demographic information (age, sex and ethnicity) and patient drug therapy information (see Table 6).

In the first phase of the study, individuals with cisplatin-induced serious hearing loss and drug-matched controls who received cisplatin but did not suffer significant hearing loss were recruited from the B.C. Children's Hospital, Vancouver, Canada. An anonymized cohort of 192 unrelated children with a clinical history of severe hearing loss that was not induced by cisplatin were recruited from the British Columbia Children's Hospital to determine the frequency of cisplatin-ototoxic genetic variants in a pediatric population with hearing impairment. The analysis of this anonymized cohort was approved by the ethics committees of the University of British Columbia and British Columbia's Children's Hospital.

A second cohort of pediatric oncology patients were recruited from across Canada. Cisplatin-induced ototoxicity was diagnosed on the basis of audiometric findings using criteria described by the CTCAE (Cancer Therapy Evaluation Program, Common Terminology Criteria for Adverse Events) Version 3. All patient data were reviewed by a clinical pharmacologist, audiologist, oncologist, and ADR surveillance clinician who reviewed audiogram test results and medical records. Patients with serious cisplatin-ototoxicity were defined as patients with ≧grade 2 CTCAE hearing impairment after treatment with cisplatin. Grade 2 to 3 hearing impairment is the point at which cisplatin pharmacotherapy protocols recommend halting or reducing cisplatin doses. Controls included pediatric oncology patients who did not develop significant hearing impairment (grade 0). The high incidence of serious ototoxicity limited the enrolment of control patients. Informed written consent was obtained from each subject and the study was approved by ethics committees of all participating universities and hospitals.

TABLE 6
Patient Demographics

| | Combined (n = 162) | | | |
|---|---|---|---|---|
| | Ototox. | | Controls | |
| | (n = 106) | | (n = 56) | |
| Age (mean, std)¹ | 6.71 | (4.51) | 8.36 | (5.41) |
| Dose (mean, std)² | 391.6 | (138.2) | 398.7 | (135.3) |
| Treatment duration (mean, std) | 5.09 | (2.79) | 5.14 | (2.71) |
| Gender (Male n, (%)) | 71 | (66.98%) | 28 | (50%) |

Concomitant medication (n, (%))

| | | | | |
|---|---|---|---|---|
| Tobramycin | 18 | (16.98%) | 10 | (17.86%) |
| Vancomycin | 13 | (12.26%) | 6 | (10.71%) |
| Vincristine | 9 | (8.49%) | 0 | |
| Gentamicin | 10 | (9.43%) | 3 | (5.36%) |

Tumor type (n, (%))

| | | | | |
|---|---|---|---|---|
| brain tumor | 25 | (23.58%) | 8 | (14.29%) |
| endodermal sinus tumor of thymus | 0 | | 1 | (1.79%) |
| germ cell tumor | 7 | (6.60%) | 15 | (26.79%) |
| hepatoblastoma | 22 | (20.75%) | 5 | (8.93%) |
| lymphoma | 0 | | 1 | (1.79%) |
| nasopharyngeal carcinoma | 1 | (0.94%) | 0 | |
| neuroblastoma | 26 | (24.53%) | 9 | (16.07%) |
| osteosarcoma | 24 | (22.64%) | 16 | (28.57%) |
| sarcoma | 1 | (0.94%) | 1 | (1.79%) |
| Cranial irradiation (n, (%)) | 23 | (21.70%) | 7 | (12.50%) |

¹Age at the start of cisplatin therapy.
²Cumulative dose received during cisplatin therapy.

### Clinical Surveillance Personnel Training

The surveillance training included ADR identification, reporting, patient enrolment, ethical issues, obtaining informed consent, advertising the project within institutions, linkage with other healthcare professionals in the institutions and data transfer.

### Ethical Approval

Ethical approval was obtained from the University of British Columbia's Clinical Research Ethics Board, the Children's and Women's Health Centre of BC ethics board as well as the local Institutional Review Board (IRB) for each clinical surveillance site.

### Biological Sample Shipping

Biological samples (5 ml of whole blood, or 2 ml of saliva, or 2 buccal swabs) were collected from each ADR case and control. Each sample was identified with a unique ID number. Blood was collected in a K2 EDTA tube following standard phlebotomy procedures at each site; samples were stored at 4° C. Saliva was collected using an Oragene™ kit (DNA Genotek™), following manufacturer's protocol; samples were stored at room temperature. Buccal swabs were collected using the BuccalAmp™ kit (Epicentre Biotechnologies™), following manufacturer's protocol; samples were stored at room temperature.

### DNA Purification

Blood samples were received and the bar-coded ID labels on the tubes were scanned to input the new samples into the genomics database. DNA was purified and stored in tubes with unique laser-etched 10-digit bar-coded labels on the bottom of the tubes, which are linked to the ID number in the database. DNA was purified from blood and buccal swabs using the Qiagen™ QiaAmp™ DNA purification kit and DNA was purified from saliva samples using the Oragene™ kit protocol.

### Genotyping

DNA samples were genotyped on the Illumina 500GX™ genotyping platform using the Illumina GoldenGate custom SNP genotyping assay to query the genotypes of 1536 single nucleotide polymorphisms (SNPs), following manufacturer's protocols (Illumina BeadStation 500G Genotyping System Manual, Illumina Document #11165222 Rev. A, 2004).

A secure database was created for storage of genotype data. This database is compatible with the raw Illumina data output.

### ADME SNP Panel

The SNP panel was developed to represent the genetic variation in 220 key ADME genes, involved in drug absorption, distribution, metabolism, elimination, drug targets, drug receptors, transporters and the like. For example, the genes include cytochrome P450 genes (CYP2D6, 2C9, 2C1 9, 3A4, 3A5, IA1), N-Acetyltransferase (NAT1, NAT2), glutathione S-transferase (GSTM1, GSTM3, GSTT1, GSTP1), histamine methyltransferase (HMT), thiopurine methyltransferase (TPMT), ATP-binding cassette, sub-family B members (ABCB1 (MDR1), ABCC1, ABCC2 (MRP1, MRP2)) nuclear receptor subfamily 1, group I, member 2 (NR 112; also called PXR or SXR) . . . .

### Identification of ADR-Associated SNPS

Case-control association tests were used to test SNP association between ADR cases and controls. An estimate of the allelic odds ratio (OR) of developing the ADR in exposed (carriers of the SNP variant) and unexposed (non-carriers of the SNP variant) patients were computed and the level of significance determined with a χ² test.

### Assessment of Ototoxicity

Ototoxicity was assessed by audiograms performed prior to initiating new therapy and prior to each subsequent dose of drug with the degree of hearing loss established using classification scheme by BROCK et al (Medical & Pediatric Oncology. 19(4): 295-300, 1991). A platinum-coordinating complex (for example, cisplatin) would be discontinued when a patient reached a grade 3 or 4 hearing loss, which affects hearing in the normal speaking frequency range. Grade 3 hearing loss is defined as marked hearing loss (>40 dB at 2000 Hz) requiring a hearing aid, and grade 4 hearing loss is defined as deafness (>40 dB at 1000 Hz or below). For this research, ototoxicity was defined as grade 3 or 4 hearing loss.

### Statistical Analysis

Hardy-Weinberg equilibrium tests are conducted using the permutation version of the exact test of Hardy-Weinberg of Guo and Thompson. Adjustments are made for multiple testing using the simpleM correction and the effective number of independent tests is calculated (MeffG) to determine significance threshold. SNPs may be removed due to HW disequilibrium and SNPs with <0.90 completion are removed for analysis. Case-control tests of association for the genotypic (2 df), allelic (1 df) and Armitage trend tests (1 df) may be performed using SAS/Genetics release 9.1 (SAS Institute Inc., Cary, N.C., USA). The average identity by state (IBS) is computed for each subject-pair, as the sum of the number of identical by state alleles at each locus divided by twice the number of loci. Principal component analysis is used to assess the population structure in the dataset.

Graphical display of principal components may be prepared with the HelixTree® software using the Eigenstrat method. Forward selection may be used in logistic regression testing for the first principal component, sex, age, cisplatin dose and treatment duration.

Homozygous and heterozygous odds ratios (OR) are calculated using the homozygous genotype of the protective allele as reference. OR computations in the presence of empty cells are adjusted by adding 0.5 to all cells. Sensitivity may be measured to assess how well the heterozygous, homozygous, or combined genotypes can correctly classify ototoxicity cases. Similarly, specificity may be measured to assess how well the genotypes can correctly classify controls. Positive predicted value (PPV) is calculated as the proportion of subjects with the ototoxicity-associated genotypes with ototoxicity, and negative predicted value (NPV) is calculated as the proportion of subjects without the ototoxicity-associated genotype and without ototoxicity.

### Linkage Disequilibrium Analysis

Additional SNPs that were in high linkage disequilibrium (LD) with the SNPs associated with cisplatin-ototoxicity were identified by scanning the 200,000 base pair region flanking each SNP of interest using the hapmap database to identify all SNPs with genotypes that were highly correlated (r²>0.7) with the genotypes of the cisplatin-ototoxicity SNPs.

### EXAMPLES

### Example 1

### Incidence of Deafness in Cisplatin-Treated Subjects

Permanent hearing loss occurs in 25% of patients receiving standard doses of cisplatin with increased severity and frequency (48%) in children less than 5 years old. Genetic variation in 220 drug metabolism genes was assessed in 106 cases of cisplatin-induced hearing loss compared to 56 drug-matched controls. Fifteen genetic variants were found to be highly predictive of cisplatin-induced hearing loss: rs1994798, rs2410556, rs11140511, rs7853758, rs4242626, rs7867504, rs4877831, rs740150, rs6464431, rs12201199, rs4646316, rs9332377, rs207425, rs3768293, and rs3101826 (see Table 7). A follow up study identified three additional genetic variants to be highly predictive of cisplatin-induced hearing loss: rs1142345, rs1800460, and rs1472408 (see Table 9).

For example, patients with the “A” variant of the “A/G” SNP “rs3101826” on chromosome 22 are susceptible to the development of Grade 3 (severe hearing loss requiring a hearing aid) or Grade 4 (deafness) hearing loss compared to patients that carry the “G” variant, which are protected from hearing loss (P=0.001).

### Example 2

### Incidence of Deafness in Cisplatin-Treated Subjects

Genetic variation in 220 drug metabolism genes was assessed in a total of 106 cases of cisplatin-induced hearing loss compared to 56 drug-matched controls (Table 6) A discovery cohort of 54 pediatric oncology patients who received cisplatin therapy was recruited from the British Columbia Children's Hospital in Vancouver, Canada. There were no significant differences in tumour types in patients with cisplatin-ototoxicity versus patients with normal hearing (see Table 6). Patients who suffered serious cisplatin-induced ototoxicity (n=33) were defined by the development of grade 2-4 hearing impairment following cisplatin therapy using CTCAE criteria, exhibiting a hearing loss of >25 dB at frequencies of 4-8 kHz. In this cohort, 22 (40%) of patients were on cisplatin treatment that did not experience any significant hearing loss (CTCAE grade 0). To better differentiate between cisplatin-ototoxicity and normal hearing, grade 1 patients were removed from the analysis. A second, independent, replication cohort of 112 pediatric oncology patients who received cisplatin pharmacotherapy were recruited from pediatric oncology units across Canada. In this cohort, 73 (66%) of the patients suffered serious cisplatin-induced ototoxicity.

TABLE 7
Fifteen genetic variants (single nucleotide polymorphisms) found to be highly predictive of cisplatin-
induced hearing loss as determined from an assessment of 220 drug metabolism genes in 106 cases
of cisplatin-induced hearing loss compared to 56 drug-matched controls (see Example 1).

| | | Geno- | Ototox. | | Controls | | | | | |
|---|---|---|---|---|---|---|---|---|---|---|
| Gene | SNP | type | (n = 106) | | (n = 56) | | OR | p-value ^{†} | Sens | Spec |
| TPMT | rs12201199 | A/_{—} | 25 | (23.6%) | 1 | (1.8%) | 17.0 | 0.000181 | 23.6% | 98.2% |
| | | T/T | 81 | (76.4%) | 55 | (98.2%) | | | | |
| COMT | rs4646316 | A/A | 1 | (0.9%) | 7 | (12.5%) | 15.0 | 0.00263 | 0.9% | 87.5% |
| | | G/_{—} | 105 | (99.1%) | 49 | (87.5%) | | | | |
| COMT | rs9332377 | A/_{—} | 31 | (29.2%) | 4 | (7.1%) | 5.4 | 0.00109 | 29.2% | 92.9% |
| | | G/G | 75 | (70.8%) | 52 | (92.9%) | | | | |
| XDH | rs207425 | A/A | 14 | (13.2%) | 0 | (0.0%) | 17.9 | 0.00421 | 13.2% | 100% |
| | | G/_{—} | 92 | (86.8%) | 56 | (100%) | | | | |
| EPHA2 | rs3768293 | C/C | 0 | (0.0%) | 12 | (23.5%) | 49.2 | 0.000025 | 0.0% | 76.5% |
| | | A/_{—} | 53 | (100%) | 39 | (76.5%) | | | | |
| MTHFR | rs1994798 | G/G | 21 | (19.8%) | 3 | (5.4%) | 4.4 | 0.0064 | 19.8% | 94.6% |
| | | A/_{—} | 85 | (80%) | 53 | (94.6%) | | | | |
| NAT2 | rs2410556 | C/C | 91 | (85.8%) | 35 | (62.5%) | 3.6 | 0.00067 | 85.8% | 37.5% |
| | | A/_{—} | 15 | (14%) | 21 | (37.5%) | | | | |
| SLC28A3 | rs11140511 | A_{—} | 102 | (96.2%) | 10 | (17.9%) | 117.3 | 0.002 | 96.2% | 82.1% |
| | | C/C | 4 | (4%) | 46 | (82.1%) | | | | |
| SLC28A3 | rs4242626 | G/G | 55 | (51.9%) | 18 | (32.1%) | 2.3 | 0.01 | 51.9% | 67.9% |
| | | A_{—} | 51 | (48%) | 38 | (67.9%) | | | | |
| SLC28A3 | rs4877831 | G/_{—} | 98 | (92.5%) | 48 | (85.7%) | 2.0 | 0.05 | 92.5% | 14.3% |
| | | C/C | 8 | (8%) | 8 | (14.3%) | | | | |
| SLC28A3 | rs7853758 | G/_{—} | 106 | (100%) | 52 | (92.9%) | Infinity | 0.005 | 100% | 7.1% |
| | | A/A | 0 | (0%) | 4 | (7.1%) | | | | |
| SLC28A3 | rs7867504 | G/G | 55 | (51.9%) | 18 | (32.1%) | 2.3 | 0.01 | 51.9% | 67.9% |
| | | A/_{—} | 51 | (48%) | 38 | (67.9%) | | | | |
| TBXAS1 | rs6464431 | A/_{—} | 54 | (50.9%) | 21 | (36.8%) | 1.8 | 0.03 | 50.9% | 63.2% |
| | | T/T | 52 | (49%) | 36 | (63.2%) | | | | |
| TBXAS1 | rs740150 | G/_{—} | 31 | (29.2%) | 11 | (19.6%) | 1.7 | 0.01 | 29.2% | 80.4% |
| | | A/A | 75 | (71%) | 45 | (80.4%) | | | | |
| SLC22A1 | rs3101826 | A/_{—} | 101 | (95.3%) | 44 | (78.6%) | 5.5 | 0.00096 | 95.3% | 21.4% |
| | | G/G | 5 | (5%) | 12 | (21.4%) | | | | |

A tiered analysis strategy identified 2 SNPs in thiopurine S-methyltransferase (TPMT) and catechol O-methyltransferase (COMT), rs12201199 and rs4646316 respectively, that were highly associated with cisplatin-induced deafness in the discovery cohort at a moderate level of significance (p<0.01), and replicated in the second cohort (p<0.01) (Table 8).

TPMT rs12201199 exhibited similar effect sizes in both the discovery and replication cohorts. The risk allele was present in 9 (27.3%) and 16 (21.9%) of the cisplatin-ototoxicity patients in the discovery and replication cohorts, while the risk allele was not present in control patients in the discovery cohort, and only 1 (2.4%) control patient in the replication cohort, conferring odds ratios of 15.9 (0.87-290.0) and 9.82 (1.25-77.37) in the discovery and replication cohorts, respectively, and was significant in a combined analysis (Fisher exact allelic test p=3.9×10-5).

TABLE 8
Genetic variants associated with cisplatin-induced hearing loss.

| | | | Combined (n = 162) | | | |
|---|---|---|---|---|---|---|
| | | Geno- | Ototox. | Controls | | |
| Gene | SNP | type | (n = 106) | (n = 56) | OR (95% CI) | p-value ^{T} |
| TPMT | rs12201199 | A/A | 3 | 0 | 4.77 (0.24, 94.11) | 0.277 |
| | | A/T | 22 | 1 | 14.94 (1.96, 114.09) | 0.000607 |
| | | A/— | 25 | 1 | 16.98 (2.23, 128.99) | 0.000181 |
| | | T/T | 81 | 55 | 1 | |
| COMT | rs4646316 | G/G | 71 | 25 | 19.88 (2.33, 169.70) | 0.000982 |
| | | G/A | 34 | 24 | 9.92 (1.14, 85.95) | 0.0215 |
| | | G/— | 105 | 49 | 15.00 (1.80, 125.29) | 0.00263 |
| | | A/A | 1 | 7 | 1 | |

The COMT ‘G’ allele of rs4646316 was present in 33 (100%) and 72 (98.6%) of the cisplatin-ototoxicity patients in the discovery and replication cohorts, while 15 (75.0%) and 34 (94.4%) of the control patients had the risk allele in the two cohorts, conferring odds ratios of 23.77 (1.24-457.45) and 4.24 (0.37-48.34). In a combined analysis, the COMT variant remained significant after correction for multiple testing (Fisher exact allelic test p=0.00034; Table 8).

The age at initiation of cisplatin therapy was slightly lower in patients developing ototoxicity (mean 6.71 years) compared to controls in both the discovery and replication cohorts (mean 8.36 years; p=0.0422; Table 6). Cisplatin-ototoxicity patients were more likely to be male (67.0%; p=0.0425). Regression analysis revealed that TPMT rs12201199 and COMT rs4646316 remained significant after adjusting for age and gender in the combined cohorts (p=0.009, p=0.0024, respectively). Additional subgroup analysis of patients >4 years of age (n=97) revealed similar associations for both TPMT and COMT (allelic tests OR:16.2, p=3.4×10-4; OR:6.3, p=0.0014, respectively).

DNA sequencing of TPMT in the patients with cisplatin-ototoxicity (n=106) and control patients without cisplatin-ototoxicity (n=56) revealed two variants, rs1800460 and rs1142345, that eliminate normal TPMT enzyme activity were present in 17 of 25 (68%) of the cisplatin-ototoxicity patients with the rs12201199 variant. Rs1800460 (Ala154Thr) was present in all 17 of these patients and rs1142345 (Tyr240Cys) was seen in 15 of these patients and none of the control patients (Table 9).

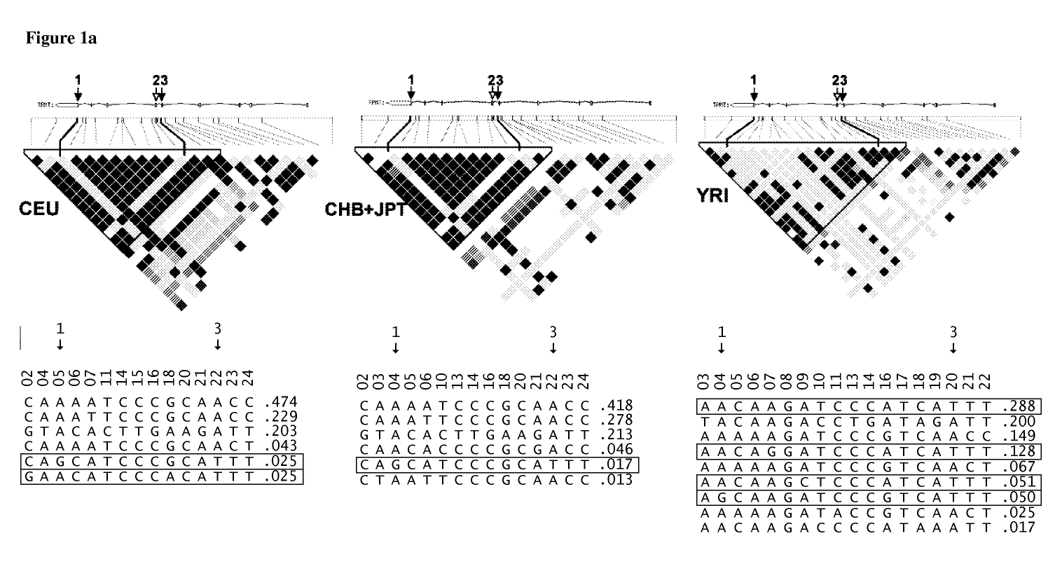

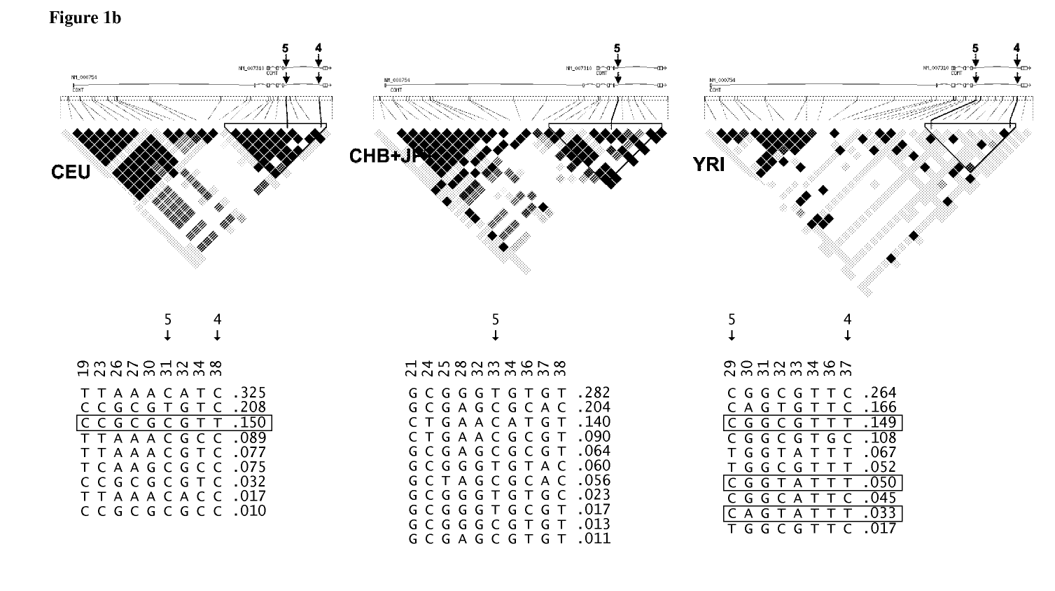

DNA sequencing of TPMT in the patients with cisplatin-ototoxicity (n=106) and control patients without cisplatin-ototoxicity (n=56) revealed an additional cisplatin-ototoxicity variant, rs9332377 (p=0.00109) in a 7.5 kb haplotype block with rs4646316 (FIG. 1b). The occurrence of TPMT and COMT risk variants in patients did not significantly overlap, and cumulatively accounted for 46 (43.4%) of the cisplatin-ototoxicity patients (Table 10).

A subgroup analysis of 107 patients that did not have the rs12201199, rs464316 or rs9332377 risk alleles (55 ototoxicity patients, and 52 controls) revealed a recessive allele of rs207425 in xanthine dehydrogenase (XDH), present only in cisplatin-ototoxicity patients in both the discovery (9.1%) and replication cohorts (15.1%) (OR: 17.90 (1.04-309.56), p=0.00421;). DNA sequencing of XDH in cisplatin-ototoxicity patients and

TABLE 9
Follow-up analysis of genetic variants associated with cisplatin-induced hearing loss.

| | | | Combined (n = 162) | | | | |
|---|---|---|---|---|---|---|---|
| | | Geno- | Ototox. | Controls | | | |
| Gene | SNP | type | (n = 106) | (n = 56) | OR (95% CI) | | p-value ^{†} |
| TPMT | rs12201199 | A/A | 3 | 0 | 4.77 | (0.24, 94.11) | 0.277 |
| | | A/T | 22 | 1 | 14.94 | (1.96, 114.09) | 0.000607 |
| | | A/_{—} | 25 | 1 | 16.98 | (2.23, 128.99) | 0.000181 |
| | | T/T | 81 | 55 | 1 | | |
| | rs1142345 (*3C) | G/G | 2 | 0 | 3.10 | (0.15, 65.79) | 0.527 |
| | | G/A | 15 | 1 | 9.27 | (1.19, 72.15) | 0.0113 |
| | | G/_{—} | 17 | 1 | 10.51 | (1.36, 81.17) | 0.00684 |
| | | A/A | 89 | 55 | 1 | | |
| | rs1800460 (*3B) | A/A | 1 | 0 | 1.82 | (0.07, 45.45) | 0.999 |
| | | A/G | 14 | 0 | 17.59 | (1.03, 300.74) | 0.00199 |
| | | A/_{—} | 15 | 0 | 18.80 | (1.10, 320.51) | 0.00131 |
| | | G/G | 91 | 55 | 1 | | |
| COMT | rs4646316 | G/G | 71 | 25 | 19.88 | (2.33, 169.70) | 0.000982 |
| | | G/A | 34 | 24 | 9.92 | (1.14, 85.95) | 0.0215 |
| | | G/_{—} | 105 | 49 | 15.00 | (1.80, 125.29) | 0.00263 |
| | | A/A | 1 | 7 | 1 | | |
| | rs9332377 | A/A | 5 | 0 | 7.65 | (0.41, 141.32) | 0.156 |
| | | A/G | 26 | 4 | 4.51 | (1.48, 13.68) | 0.00524 |
| | | A/_{—} | 31 | 4 | 5.37 | (1.79, 16.14) | 0.00109 |
| | | G/G | 75 | 52 | 1 | | |
| XDH | rs207425 | A/A | 14 | 0 | 17.90 | (1.04, 309.56) | 0.00421 |
| | | A/G | 35 | 21 | 1.02, | (0.52, 2.03) | 0.999 |
| | | A/_{—} | 49 | 21 | 1.43 | (0.74, 2.78) | 0.318 |
| | | G/G | 57 | 35 | 1 | | |
| EPHA2* | rs3768293 | A/A | 30 | 15 | 49.19 | (2.73, 887.03) | 0.0000246 |
| | | A/C | 23 | 24 | 23.98 | (1.34, 428.40) | 0.00181 |
| | | A/_{—} | 53 | 39 | 33.86 | (1.95, 589.19) | 0.0000918 |
| | | C/C | 0 | 12 | 1 | | |
| | rs1472408 | A/A | 31 | 15 | 20.67 | (2.42, 176.73) | 0.000591 |
| | | A/G | 22 | 27 | 8.15 | (0.97, 68.66) | 0.0387 |
| | | A/_{—} | 53 | 42 | 12.62 | (1.55, 102.55) | 0.00363 |

| | | | |
|---|---|---|---|
| G/G | 1 | 10 | 1 |

^{†} Fisher exact test in combined cohorts.
*EPHA2 genotype results for patients without the TPMT or COMT risk variants.

controls revealed a novel, predicted loss-of-function, truncation variant (R881X) in a single patient with grade 3 severe hearing impairment.

This subgroup analysis also revealed a significant association for a protective variant in the ephrin receptor A2 (EPHA2), rs3768293, in 4 (23.5%) of the controls in the discovery cohort and 8 (23.5%) of the controls in the replication cohort, and was not in any ototoxicity patients (OR: 0.03 (0.002-0.51); Fisher exact p=2.46×10-5). A second variant in EPHA2, rs1472408, in LD with rs3768293 was also significant (OR:0.08 (0.01-0.64), Fisher exact p=5.91×10-4; Table 9).

TABLE 10
Genotype-driven prediction of cisplatin ototoxicity.

| | | | Combined (n = 162) | | | | | | | |
|---|---|---|---|---|---|---|---|---|---|---|
| | | Geno- | Ototox. | Controls | | | | | | |
| Gene | SNP | type | (n = 106) | (n = 56) | OR | p-value ^{†} | Sens | Spec | PPV | NPV |
| TPMT | rs12201199 | A/_{—} | 25 (23.6%) | 1 (1.8%) | 17.0 | 0.000181 | 23.6% | 98.2% | 96.2% | 40.4% |
| | | T/T | 81 (76.4%) | 55 (98.2%) | | | | | | |
| COMT | rs4646316 | A/A | 1 (0.9%) | 7 (12.5%) | 15.0 | 0.00263 | 0.9% | 87.5% | 12.5% | 31.8% |
| | | G/_{—} | 105 (99.1%) | 49 (87.5%) | | | | | | |
| COMT | rs9332377 | A/_{—} | 31 (29.2%) | 4 (7.1%) | 5.4 | 0.00109 | 29.2% | 92.9% | 88.6% | 40.9% |
| | | G/G | 75 (70.8%) | 52 (92.9%) | | | | | | |
| Unique carriers of either¹ | | | 46 (43.4%) | 1 (1.8%) | 42.2 | 1.1 × 10⁻⁹ | 43.4% | 98.2% | 97.9% | 47.8% |
| Non-carriers | | | 60 (56.6%) | 55 (98.2%) | | | | | | |
| XDH | rs207425 | A/A | 14 (13.2%) | 0 (0.0%) | 17.9 | 0.00421 | 13.2% | 100% | 100% | 37.8% |
| | | G/_{—} | 92 (86.8%) | 56 (100%) | | | | | | |
| Unique carriers of either² | | | 57 (53.8%) | 1 (1.8%) | 64.0 | 5.65 × 10⁻¹³ | 53.8% | 98.2% | 98.3% | 52.9% |
| Non-carriers | | | 49 (46.2%) | 55 (98.2%) | | | | | | |
| EPHA2* | rs3768293 | C/C | 0 (0.0%) | 12 (23.5%) | 49.2 | 0.000025 | 0.0% | 76.5% | 0.0% | 42.4% |
| | | A/_{—} | 53 (100%) | 39 (76.5%) | | | | | | |

| Cumulative total³ | 57 (53.8%) | 1 (1.8%) | 64.0 | 5.65 × 10⁻¹³ | 53.8% | 98.2% | 98.3% | 52.9% |
|---|---|---|---|---|---|---|---|---|
| | 49 (46.2%) | 55 (98.2%) | | | | | | |

^{†} Fisher exact test in combined cohorts.
*EPHA2 genotype results for patients that did not carry the TPMT or COMT risk variants.
¹Cumulative subtotal of unique patients with TPMT rs12201199 A_, or COMT rs9332377 A_ or rs4646316 A/A cisplatin-ototoxicity susceptibility variants.
²Cumulative subtotal of TPMT, COMT, or XDH rs207425 A/A susceptibility variants.
³Cumulative total of all TPMT, COMT, XDH, or EPHA2 rs3768293 C/C cisplatin-ototoxicity susceptibility variants.
Sensitivity (Sens), Specificity (Spec), PPV (positive predictive value), NPV (negative predictive value).

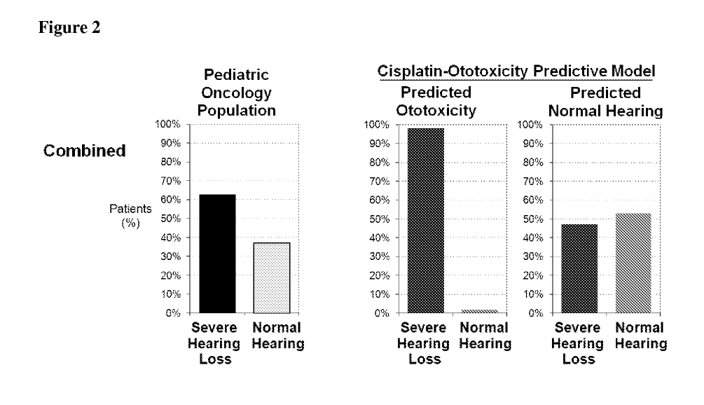

The combination of the identified cisplatin-ototoxicity associated variants was used to develop a genotype-based model to predict the occurrence of cisplatin-induced ototoxicity in the discovery (OR:62.25 (3.47-1018.40), p=3.84×10-6) and replication cohorts (OR:35.97 (4.68-267.67), p=1.14×10-7) (Table 10). In the combined cohorts, a genetic test of variants in TPMT, COMT, XDH, and EPHA2 identified 53.8% (sensitivity) of the cases of severe hearing loss in children that received cisplatin pharmacotherapy, with an accuracy of predicted severe hearing loss of 98.3% (positive predictive value), and specificity of 98.2% (OR: 63.98 (8.54-479.54), p=5.65×10-13) (FIG. 2). Three of the ototoxicity susceptibility SNPs (rs12201199, rs9332377, and rs207425), individually can correctly identify 23.6%, 29.2%, and 13.2%, respectively, of the cases of cisplatin ototoxicity (i.e. the sensitivity). Additionally, each of these SNPs have varying rates of false positives: 1.8%, 7.1%, and 0% (i.e. specificity). Furthermore, rs4646316 can correctly identify 12.5% of patients protected from cisplatin ototoxicity. Additionally, each of these SNPs have varying rates of false positives: 1.8%, 7.1%, 0%, 0.9% (i.e. specificity). In combination, however, the overall ability to correctly identify cases of cisplatin ototoxicity are significantly improved. Combining rs12201199 and rs9332377 increases the specificity to 43.4%, with a false positive rate of 1.8%. Combining rs12201199, rs4646316, rs9332377, and rs207425 together further increases the sensitivity to 53.8%, with a false positive rate of 1.8%.

Using the Eigenstrat principal component analysis, we found that the majority of the patients (85%) were of European ancestry. In a subgroup analysis of only European ancestry patients (n=145), the TPMT variants remained highly associated with cisplatin-ototoxicity and the associations became stronger for COMT, XDH, and EPHA2.

What is claimed is:

1. A method of treating a human subject, the method comprising:
(a) determining whether the subject has a risk allele selected from one or more of the following: rs1142345G; rs12201199A; rs1800460A; rs9332377A; rs1994798G; rs2410556G; rs4242626G; rs7867504G; rs11140511A; rs4877831G; rs7853758G; rs740150G; rs6464431A; rs3101826A; rs207425A; rs3768293A; and rs1472408A; or
a reduced risk allele selected from one or more of the following: rs1142345A; rs12201199T; rs1800460G; rs9332377G; rs1994798A; rs2410556A; rs4242626G; rs7867504A; rs11140511C; rs4877831C; rs7853758A; rs740150A; rs6464431T; rs3101826G; rs207425G; rs3768293C; and rs1472408G;
(b) selecting a treatment regimen based on the subject's ototoxicity risk status, wherein subjects with reduced risk are administered the platinum-coordinating compound, and wherein subjects with one or more risk alleles are provided with two or more of the following:
(i) platinum coordinating compound administration;
(ii) hearing acuity monitoring;
(iii) non-platinum coordinating anti-neoplastic compound administration; and
(iv) otoprotectant administration;
(c) administering the treatment regimen selected in (b).

2. The method of claim 1, wherein the platinum-coordinating compound is selected from one or more of the following: cisplatin; carboplatin; oxaliplatin; tetraplatin; ormiplatin; iproplatin; and satraplatin.

3. The method of claim 1, wherein the determining of the identity of a single nucleotide polymorphism is by one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

4. The method of claim 1, wherein the human subject is a human pediatric subject.

5. The method of claim 1, further comprising determining the identity of rs4646316 as a risk allele G or a reduced risk allele A.

6. A method of treating a human pediatric subject, the method comprising:
(a) determining whether the subject has a risk allele selected from one or more of the following: rs1142345G; rs12201199A; rs1800460A; rs9332377A; rs1994798G; rs2410556G; rs4242626G; rs7867504G; rs11140511A; rs4877831G; rs7853758G; rs740150G; rs6464431A; rs3101826A; rs207425A; rs3768293A; and rs1472408A; or
a reduced risk allele selected from one or more of the following: rs1142345A; rs12201199T; rs1800460G; rs9332377G; rs1994798A; rs2410556A; rs4242626G; rs7867504A; rs11140511C; rs4877831C; rs7853758A; rs740150A; rs6464431T; rs3101826G; rs207425G; rs3768293C; and rs1472408G;
(b) selecting a treatment regimen based on the subject's ototoxicity risk status, wherein subjects with reduced risk are administered the platinum-coordinating compound, and wherein subjects with one or more risk alleles are provided with two or more of the following:
(i) platinum coordinating compound administration;
(ii) hearing acuity monitoring;
(iii) non-platinum coordinating anti-neoplastic compound administration; and
(iv) otoprotectant administration;
(c) administering the treatment regimen selected in (b).

7. The method of claim 6, wherein the platinum-coordinating compound is selected from one or more of the following: cisplatin; carboplatin; oxaliplatin; tetraplatin; ormiplatin; iproplatin; and satraplatin.

8. The method of claim 6, wherein the determining of the identity of a single nucleotide polymorphism is by one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

9. The method of claim 6, further comprising determining the identity of rs4646316 as a risk allele G or a reduced risk allele A.

10. A method of treating a CNS tumor, a hepatoblastoma, or an osteosarcoma in a human subject, the method comprising:
(a) determining whether the subject has a risk allele selected from one or more of the following: rs1142345G; rs12201199A; rs1800460A; rs9332377A; rs1994798G; rs2410556G; rs4242626G; rs7867504G; rs11140511A; rs4877831G; rs7853758G; rs740150G; rs6464431A; rs3101826A; rs207425A; rs3768293A; and rs1472408A; or
a reduced risk allele selected from one or more of the following: rs1142345A; rs12201199T; rs1800460G; rs9332377G; rs1994798A; rs2410556A; rs4242626G; rs7867504A; rs11140511C; rs4877831C; rs7853758A; rs740150A; rs6464431T; rs3101826G; rs207425G; rs3768293C; and rs1472408G;
(b) selecting a treatment regimen based on the subject's ototoxicity risk status, wherein subjects with reduced risk are administered the platinum-coordinating compound, and wherein subjects with one or more risk alleles are provided with two or more of the following:
(i) platinum coordinating compound administration;
(ii) hearing acuity monitoring;
(iii) non-platinum coordinating anti-neoplastic compound administration; and
(iv) otoprotectant administration;
(c) administering the treatment regimen selected in (b).

11. The method of claim 10, wherein the method further comprises obtaining a nucleic acid sample and assaying the sample prior to step (a).

12. The method of claim 10, wherein the platinum-coordinating compound is selected from one or more of the following: cisplatin; carboplatin; oxaliplatin; tetraplatin; ormiplatin; iproplatin; and satraplatin.

13. The method of claim 10, wherein the determining of the identity of a single nucleotide polymorphism is by one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

14. The method of claim 10, wherein the human subject is a human pediatric subject.

15. The method of claim 10, further comprising determining the identity of rs4646316 as a risk allele G or a reduced risk allele A.

16. The method of claim 1, wherein the otoprotectant is selected from one or more of: a xanthine dehydrogenase inhibitor; sodium thiosulfate; ebselen; d-methionine; glutathione ester; diethyldithiocarbamate; amifostine; tiopronin; α-tocopherol; salacylate; aminoguanidine; trolox; Z-DEVD-fluoromethyl ketone; ZLEKD-fluoromethyl ketone; 2-chloro-N-cyclopentyladenosine; pifithrin; α-lipoic acid; deferoxamine; 2,2′-dipyridyl; salicylate; 2,3-dihydroxybenzoate; dexamethasone; TRANSFORMING GROWTH FACTOR-β1; GLIAL-CELL-DERIVED NEUROTROPHIC FACTOR; ethacrynic acid; CEP1347; or minocycline.

17. The method of claim 6, wherein the otoprotectant is selected from one or more of: a xanthine dehydrogenase inhibitor; sodium thiosulfate; ebselen; d-methionine; glutathione ester; diethyldithiocarbamate; amifostine; tiopronin; α-tocopherol; salacylate; aminoguanidine; trolox; Z-DEVD-fluoromethyl ketone; ZLEKD-fluoromethyl ketone; 2-chloro-N-cyclopentyladenosine; pifithrin; α-lipoic acid; deferoxamine; 2,2′-dipyridyl; salicylate; 2,3-dihydroxybenzoate; dexamethasone; TRANSFORMING GROWTH FACTOR-β1; GLIAL-CELL-DERIVED NEUROTROPHIC FACTOR; ethacrynic acid; CEP1347; or minocycline.

18. The method of claim 10, wherein the otoprotectant is selected from one or more of: a xanthine dehydrogenase inhibitor; sodium thiosulfate; ebselen; d-methionine; glutathione ester; diethyldithiocarbamate; amifostine; tiopronin; α-tocopherol; salacylate; aminoguanidine; trolox; Z-DEVD-fluoromethyl ketone; ZLEKD-fluoromethyl ketone; 2-chloro-N-cyclopentyladenosine; pifithrin; α-lipoic acid; deferoxamine; 2,2′-dipyridyl; salicylate; 2,3-dihydroxybenzoate; dexamethasone; TRANSFORMING GROWTH FACTOR-β1; GLIAL-CELL-DERIVED NEUROTROPHIC FACTOR; ethacrynic acid; CEP1347; or minocycline.

19. The method of claim 16, wherein the xanthine dehydrogenase inhibitor is such as allopurinol or fosfomycin.

20. The method of claim 17, wherein the xanthine dehydrogenase inhibitor is such as allopurinol or fosfomycin.

21. The method of claim 18, wherein the xanthine dehydrogenase inhibitor is such as allopurinol or fosfomycin.


SEQUENCE LISTING (18 sequences)

SEQ ID NO: 1 - DNA, 360 bp, Homo sapiens
ccttgtctcaattctctgtccccatcctcacccaggcgtcccctaccctgggctctcagcgcccaccccaagcgccgagaggaagatgtacgtcccatcttctgggcctccagaccaaagagttacatctaccgtacccaggagtgggacgagttccctaacggccgctggtgagggcctgcagaccttccttgcaaatayatctttgttcttgggagcgggagggcagaagaagtttgcatgcttgtggttgacctgggaggagtcaggggcagaatttacaggaatggcctcctgggcatgtggtggcactgccctctgtcaggagtgtgccctgacctctgggcacccctctgccag

SEQ ID NO: 2 - DNA, 420 bp, Homo sapiens
gctatctgtaaaaaaatacgtttaacattaaggtttatacttgggtgaacacctgatattcacaggctataaaatagttagcaaggaaataactttaaatgtgactagttttgtctaatgtctcagttctcaaagcaatctaggtaaactgctaacaatgaataaattgaacaaatataagtgggatggataaatgcttgytggttaacttttatgtaatttaaaatcttaaacttattttggattaaagaacagctactcattaatagtttggctcatttccaattaagtagagatatggagaaacatgcctaaaaattatagagtgatttcatctataaagtactgatacctgatatgcagtttaggatttcatgtttcctaggtttaaggtcactaaaaataaaaattccacttaat

SEQ ID NO: 3 - DNA, 420 bp, Homo sapiens
acatgctcctgggaacagagcaggggagatggatatctaaccaaagctgaaatagtcacaggaagagagtgtttctggcacactatgagatcctctgttagcttttaaccaatgtttattacgtgtttattgttagaaaataaaaagccaaggaactctgtcatccctcttggtttggcaaagtttgagcaagttggtggygctctgtcccccatcaccatccccgttagtccaaaactgatggacctcatggggtgtgcttaaaatgcaaagtaggatttcctggatgttagggctattaaccaatgggttgtcacagctttctcagaaagctctggagttgttggaatgtctttatttccatccagggctttgttatgggctgggtgggtagtgtgtgagtaatgtgtaggttgggtc

SEQ ID NO: 4 - DNA, 420 bp, Homo sapiens
cccccgatgattcagtgaggtcccagagaaaataaaggtttaatctttttccacataaagttaatatttgggggaatccctgaaaaaaaaaaaaaaaaagacagtggtatccctaaccatctttgttatttacctgctaataaaatgccccagatgatgtgccgaagagttgttttgtgtttcctacagaaaccacatacygtgtcatacctcctttccaaacacctgcaacataaaagcaaaaaggcagggagaagtaaacaccaaaaacatgaaataaactgcatggtgaggctagattaagctttgtaaactaagaaggcaaacaaacacatgtgtgaggcgtatgtcttacaaaatagaaatgggaaactcatttcattttagactagagtcagccatgatcatgaccattcagtg

SEQ ID NO: 5 - DNA, 401 bp, Homo sapiens
ggtaggagaattgcttgagccccagaggcagatcatgccactgcactccagcctgggagacacagcaagactctacctgaaaaaaaaaaaaaagaaaagaaaagaaagtaaagaaaagaaacatatgacttttgctttgttttgttctgatacaaaagtggtccaggggaagagaggaatgaggacaattgccctcatctmggcctgataactttccaataggcagagctgggaccaggggcaggtttccactctcctttctatcacatcacactgattttcaacttataaaatgaactaaaacgtggtaaaccataggctgaatagccttattcttagatgttagatttacgaaatgttgaaatgggttttttttggtttaaagcaatctctggcagtaa

SEQ ID NO: 6 - DNA, 420 bp, Homo sapiens
ttgtctcaaggctttcagatcacagactctgagccttttctctccctcatccacgtggagagcagtgatgatgctacactaactctcgaggatgtccaggcatgttacttttggacagtttctcagagctcacagaatccattaagttcaaagggagagtgctttaagatcactagactgttagccactagactatgaggscaggggccccatgacttgctatggcctcagtactcagcgcattgcacataaaaggcattcaaaaaattttggttgattgattgcagtcccagctcatgtgactctgggtctgtgtccacagtcctcaaaagtagatgattatagtggcaatgattagctgtgtctacacacacacgcacactctaattcaacataactgaaagttaggaagtgctatgt

SEQ ID NO: 7 - DNA, 390 bp, Homo sapiens
gggatgcatgagaagcttctacggtgtggaagagtctactgaggttagggtgggctgtttacaaacctattttatttttaaacaaagataggcagaaacaaaacagagggcaggggcgtgatgtgattatacctcaaaactcagctgtgggtagtcaaacatgtttccaaaccaggacagggctgaattcataaaagacarcagggccaggaaggcaatcagattcacagcgatgttggccaccagggagatggaggaggatgctccctgtgttgcagcttctagaagattccctgaatcactttatcaagaaatagcaattccagaattaccaaggagttgtcaggggatggacaccattggtgcagaagtagcataatcagagcttag

SEQ ID NO: 8 - DNA, 420 bp, Homo sapiens
gggatttcagggctgcctgctggggagggaggcttgaagctgtgtctgcactgcatcttcacagcactgagaaaatgccagtcattcaggaggacagggcagccctgtcctcgccacagtgcccgcatcttcattggttcacctttgcaggtatcatctgtgacctgtcatccaggctctgagccctgaggataataagtytccctcgaaggtcctgggtttgttggagctttttctccctcttgccatgtccttttcacggtccatcctctgagtttgtgaattatctttcttcgtggcccatgtggcacaagatgaagagtgcatgggcagcttctcccctgacatccctacctccctgacgcttaacccacataggggaagaacattcctgaaacagagacgtcacccaaggttgct

SEQ ID NO: 9 - DNA, 390 bp, Homo sapiens
gaaatagtttacttcaccattgtgaattatcaacattacaaccagtaattactaattacctccattttgagttgatgttttgggggatgtttttatcctgcagtgctatggagaggctgggaacgcactgctgcctgtgttgcttgtttatttatttccttttattcccagtgttctctcattccccttcccatccccaawagtcaaccattccaattgtgtttaatgtgtttttttttcacatgttcttgcaaattgtgcattatcatttcgtggatattatccttagtttatgccaatagtactgccgtagatctcattctattccttagttttttcatttagcactgtgctttcaaggtccagcacagagttcccattgagtctgct

SEQ ID NO: 10 - DNA, 390 bp, Homo sapiens
tgaaattcatttctattacaggcccaggtgcaaggtagaagacactgtcttactcacctttctttttccctagctgcctcagtttcccatagtttgggagctaaccaaagacaaaacacattaaagtgtgcagacgagtgtgtaataaaaatatctgcagaacagacattcaaaaaaatgctttgtggatgttacacaggwggaagagagtgaggaagacacctccactcccatgcctgcactgcctggcaagcattcaaattttttaaagtgcagatgtagtattcaacctacctgggaagatcaaaaatactgcaacagtacaatgaaatgttccccgaagaactctgtaatgaaataatgaaaaaaaaattttttttttttacttag

SEQ ID NO: 11 - DNA, 420 bp, Homo sapiens
cctgggaaagaagtttcagtatctcctgtgtgttctttcttatgatccaactaaacatccaggtccaccattttatgttccacatgctgaaattgaaaggttgtttggtaaaatatgcaatatacgttgtcttgagaaggttgatgcttttgaagaacgacataaaagttggggaattgactgtctttttgaaaagttatrtctacttacagaaaagtaaatgagacatagataaaataaaatcacactgacatgtttttgaggaattgaaaattatgctaaagcctgaaaatgtaatggatgaatttttaaaattgtttataaatcatatgatagatctttactaaaaatggctttttagtaaagccatttactttttctaaaaaagttttagaagaaaaagatgtaactaaactttta

SEQ ID NO: 12 - DNA, 390 bp, Homo sapiens
tccacacccaggtccacacattcctctaggaggaaacgcagacgtgagatcctaataccttgacgattgttgaagtaccagcatgcaccatgggggacgctgctcatcttcttaaagatttgatttttctcccataaaatgttttttctctttctggtaggacaaatattggcaaatttgacatgatttgggatagaggarcattagttgccatcaatccaggtgatcgcaaatggtaagtaatttttctttttttgtttagctgtcttaattttttagtatactatactttttctgggttctagaaaatcagcttagacttctatgagtttgaaataggttattatgtttggaatttataaaaacctaaatccaatactagctttgtct

SEQ ID NO: 13 - DNA, 420 bp, Homo sapiens
agctcgctctggaggcaccacctgaggtctgggagtgtgggggactgaggaggccctgtggtgggtggagatgggtggggagctgggccaggggcctggctgggtggcctgttgggaactggggagccagctgcctgtgcaggtgcaaaatgggtggcagaagtggggtgcacaccccagaccagacaccagggcagaaayggcacaggaccaaggagatggggtggggaagggccgctctgggcccagcctgctctcccccaagcaagccactgctcgtgcaaagaaagcatgtgtctcctgcagatcttcctcctgaggccccatcttgtgcattcccccaacccagccccactggcgaggaccctgagtgccccgagtgaggctagacagcgggtggggctgtcctcgcttccctgg

SEQ ID NO: 14 - DNA, 420 bp, Homo sapiens
ctgtgtcctcccagggcccaggcactggtgaagatggggggtctgcaaatgcaggagcttggggatgtccagaactgaccccaaggggcaggcttgttgatgggaggtctgccccacctcagccctgcagggtcaccctggtcaggccaatattgtctccagggaccataccagcaacccctctccttgggtgcctctccytcataggcctgagttcctggcactgggtgttgagggccccattgtttccactcacccagctagcatttattgagcacctactgtgtgccacatgctgttctaagggatggatactcctgagatggatacaggagttgatgagagaaaggtccctgtcctcacggggcccatgttctgaaggtggcacccaagtcttgtacagtcctttcctgcaggagt

SEQ ID NO: 15 - DNA, 387 bp, Homo sapiens
tatttttagtctttttaatgttagtcattgtggtggttaatgggtgtgaattttaaaacacatacgctgtgcactgcttaggatatgttcacatgcattaaaatataaaaacatggatgggaatgttttacatcaaccttaggatggtaattacctcaaggcagagggcaaaatctcagagtggggtgataaaatgggaarcttcaactgtacttgtaacattaatcttttaccatgggaaacaaatatgggcaaacaaacatttttttagaaccaggtgggaagtatatatgtgtattttatcgttctctatattacttatgtttggttcatactgtaattttctttttatttaagaaaaatgtttttaaaggcaaaaatagaccc

SEQ ID NO: 16 - DNA, 420 bp, Homo sapiens
tgggctgtgggggtttatggcccctgcctggcctgagagcctgggctgggaacgcccccgagcttcccaagtcagctggccctggaccacctgaaccagcactgagccaggtgaagtctcctccacagacaagtcagggcatttgggggaactgaccccaggataaacatggcccagctttctggagtctcagttttactmacagatctggtggaggagagagctatttttgtatcatgcagatatgttatggggagggatgcaatatactcatgggtttccaaatacaataggagacttgaagacattcggggcttgaaggagaccacttcagccaccccaaccaacccccctgggctggagtctacacttttctgctcacagacacaaaagtgcccagtttgagaagatctgtggtgg

SEQ ID NO: 17 - DNA, 420 bp, Homo sapiens
ctgggggccttcagcccaggtaggagggccatgtcacttcaggaggcggtcttcaagaccaccctcagagcccagctcccatctccacaaaccaggccatccctgctcccagcctgcctggagctctgtccaccctttgagtccttctccggtcctggccttgaggaatggggcttctgaggcagatccctcatgctccarggcccaaaggaagcattgacttggtttctttacccccaccttagggctttaccctctgaatccatcttgcataggttctatgccccggtttctcctatctccttaccctctagggagggtagcacttattggcagctacctggactttacttggaaatagagtggggacagtacctagggtcttaggttttggctatggcctctgagcctggcagagaa

SEQ ID NO: 18 - DNA, 390 bp, Homo sapiens
atcaagttcctctggaatcactcctatcagtggcaaagcggtggacaccccacgactatctccgccattgtcaggaatttcaggagcttcattggctaagcctgcagcactcagggtgacccggctggaaggcagagtagtcgagaatcattcttttggagacaagactgaaaaggcttccctggctggtctgagcaggaygttctaagggtcgctgctccttggtggtgtgagaagcacattctctttggaactgcagtaactaagcacctagctgcaactagggctatggtgagtttgcctcgattattgttaaattgcagtttacctgacacctcacttgtgatttagtttaaaaattttaaattaccaagaaacgggggaaaaaaa